SEQUENCE LISTING (10 sequences)

SEQ ID NO: 1 - protein, 327 aa, Erwinia chrysanthemi
ADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 2 - protein, 20 aa, Artificial Sequence
  Synthetic Construct
AAPAAPAPAAPAAPAPAAPA

SEQ ID NO: 3 - protein, 201 aa, Artificial Sequence
  Synthetic Construct
AAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAA

SEQ ID NO: 4 - protein, 400 aa, Artificial Sequence
  Synthetic Construct
AAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPA

SEQ ID NO: 5 - protein, 326 aa, Escherichia coli
LPNITILATGGTIAGGGDSATKSNYTAGKVGVENLVNAVPQLKDIANVKGEQVVNIGSQDMNDDVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKTVFDTLATAAKNGTAVVRSSRVPTGATTQDAEVDDAKYGFVASGTLNPQKARVLLQLALTQTKDPQQIQQIFNQY

SEQ ID NO: 6 - protein, 348 aa, Escherichia coli
MEFFKKTALAALVMGFSGAALALPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 7 - protein, 326 aa, Escherichia coli
LPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 8 - protein, 327 aa, Dickeya chrysanthemi; identical to SEQ ID NO: 1

SEQ ID NO: 9 - protein, 528 aa, Artificial Sequence
  Synthetic Construct
AAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 10 - protein, 728 aa, Artificial Sequence
  Synthetic Construct
AAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

(19) United States
(12) Patent Application Publication
(10) Pub. No.: US 2022/0227805 A1
(43) Pub. Date: Jul. 21, 2022

(54) METHODS OF PURIFYING CHARGE-SHIELDED FUSION PROTEINS

(71) Applicants: Jazz Pharmaceuticals Ireland Ltd., Dublin (IE); Pfenex, Inc., San Diego, CA (US)

(72) Inventors: Christopher Kable MEANS, Encinitas, CA (US); Shahparak ZALTASH, Solana Beach, CA (US); Nina MP STELZER, San Diego, CA (US)

(21) Appl. No.: 17/559,978

(22) Filed: Dec. 22, 2021

Related U.S. Application Data
(60) Provisional application No. 63/130,295, filed on Dec. 23, 2020.

(51) Int. Cl. C07K 1/20; C07K 1/18; C07K 1/36; C12N 9/82; B01D 15/32; B01D 15/36; B01D 15/42

(52) U.S. Cl. CPC C07K 1/20; C07K 1/18; C07K 1/36; C12N 9/82; C12Y 305/01001; A61K 38/00; B01D 15/363; B01D 15/362; B01D 15/424; C07K 2319/31; B01D 15/327

(57) ABSTRACT

The present invention relates to method of purifying charge-shielded proteins from a cell lysate or periplasmic releasate using hydrophobic interaction chromatography as a first chromatography steps. Also provided herein are compositions comprising charge-shielded proteins and methods of treatment using purified charge-shielded proteins.

### CROSS-REFERENCE TO RELATED APPLICATIONS

This application claims priority to U.S. Application No. 63/130,295, filed Dec. 23, 2020, the contents of which are incorporated in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 210462000100SEQLIST.TXT, date recorded: Dec. 9, 2021, size: 30,160 bytes).

### FIELD

The present invention relates to methods of purifying charge-shielded fusion proteins.

### BACKGROUND

Many proteins of pharmaceutical interest, in particular certain enzymes and recombinant antibody fragments, hormones, interferons, etc. suffer from rapid (blood) clearance. This is particularly true for proteins whose size is below the threshold value for kidney filtration of about 70 kDa (Caliceti (2003) Adv Drug Deliv Rev 55:1261-1277). In these cases the plasma half-life of an unmodified pharmaceutical protein may be one the order of a few hours, thus rendering it essentially useless for most therapeutic applications. In order to achieve sustained pharmacological action and also improved patient compliance—with required dosing intervals extending to several days or even weeks—several strategies were previously established for purposes of biopharmaceutical drug development.

One methodology for prolonging the plasma half-life of biopharmaceuticals is the conjugation with highly solvated and physiologically inert chemical polymers, thus effectively enlarging the hydrodynamic radius of the therapeutic protein beyond the glomerular pore size of approximately 3-5 nm (Caliceti (2003)). Thus fusion proteins have been developed which comprises biologically active domain and an additional domain that increases the hydrophobic radius of the fusion protein without affecting the biologically activity of the biologically active domain.

However, production and purification of such fusion proteins present challenges necessitating new purification methods. In particular, prior to the present invention, it was not known that fusion of a domain to increase the hydrodynamic radius of a biologically active domain could cause a charge-shielding effect thus making conventional purification methods unsuitable for such fusion proteins. The present inventors identified the charge-shielding effect and novel methods to purify such therapeutic fusion proteins.

### BRIEF SUMMARY

Provided herein are methods of purifying charge-shielded proteins from a cell lysate or periplasmic releasate. In some embodiments, the method comprises a hydrophobic interaction chromatography as a first chromatography step. In some embodiments, the method comprises an anion exchange chromatography as a second chromatography step. In some embodiments, the method comprises a cation exchange chromatography as a third chromatography step.

In some embodiments, provided herein is a method of purifying a charge-shielded fusion protein from a cell lysate or periplasmic releasate, wherein the charge-shielded fusion protein comprises a biologically active domain and a charge-shielding domain, and wherein the method comprises hydrophobic interaction chromatography as a first chromatography step.

In some embodiments, provided herein is a method for producing a charge-shielded fusion protein from a cell lysate or periplasmic releasate wherein the charge-shielded fusion protein comprises a biologically active domain and a charge-shielding domain, wherein the method comprises i) culturing cells comprising a nucleic acid encoding the charge-shielded fusion protein; and ii) purifying the charge-shielded fusion protein, wherein the charge-shielded protein is purified from the cell lysate or periplasmic releasate using hydrophobic interaction chromatography as a first chromatography step.

In some embodiments, the charge-shielded fusion protein is at least 45% pure after the first chromatography step. In some embodiments, the method further comprises an anion exchange chromatography. In some embodiments, the method further comprises a cation exchange chromatography.

In some embodiments, the method comprises a sequence of chromatography steps comprising in order i) hydrophobic interaction chromatography; ii) anion exchange chromatography; and iii) cation exchange chromatography.

In some embodiments, the biologically active domain is charged at pH of about 7.0, and wherein the charge-shielding domain increases the hydrodynamic radius of the protein, and wherein the charge-shielding domain does not have a charge at pH of about 7.0. In some embodiments, the molecular weight of the biologically active domain is less than the molecular weight of the charge-shielding domain. In some embodiments, the molecular weight of the charge-shielding domain is between 10 kDa and 60 kDa. In some embodiments, the molecular weight of the charge-shielding domain is between 10 kDa and 20 kDa. In some embodiments, the molecular weight of the biologically active domain is between 30 kDa and 40 kDa. In some embodiments, the molecular weight of the charge-shielding domain is sufficient to increase the in vivo half-life of the charge-shielded fusion protein or a multimer of the charge-shielded fusion protein. In some embodiments, the in vivo half-life of the charge-shielded fusion or a multimer of the charge-shielded protein is increased compared to the half-life of a protein comprising the biologically active domain or a multimer of a protein comprising the biologically active domain without the charge-shielding domain.

In some embodiments, the charge-shielding domain has a random coil or disordered structure. In some embodiments, the charge-shielding domain is a polypeptide consisting of one or more of alanine, serine and proline residues. In some embodiments, the charge-shielding domain is a polypeptide consisting of proline and alanine residues.

In some embodiments, the method comprises purifying a PASylated biologically active fusion protein from a cell lysate or periplasmic releasate comprising i) culturing cells comprising a nucleic acid encoding the PASylated biologically active protein; and ii) purifying the PASylated biologically active protein, wherein the PASylated biologically active protein is purified from the cell lysate or periplasmic releasate using hydrophobic interaction chromatography as a first chromatography step.

In some embodiments, provided herein is a method for purifying a charge-shielded fusion protein comprising a biologically active domain and a charge-shielding domain from a cell lysate or periplasmic releasate, the method comprising the following steps in order i) applying a load solution comprising the charge-shielded fusion protein to a hydrophobic interaction chromatography column; ii) applying a wash solution to the hydrophobic interaction chromatography column; iii) applying an elution solution to the hydrophobic interaction column to elute the charge-shielded protein; iv) applying the eluted charge-shielded fusion protein in iii) as a load solution to an anion exchange chromatography column; v) eluting the charge-shielded fusion protein from the anion exchange chromatography column; vi) applying the eluted charge-shielded fusion protein in vi) as a load solution to a cation exchange chromatography column; vii) applying a wash solution to the cation exchange chromatography column; viii) applying an elution solution to the cation exchange chromatography column to elute the charge-shielded fusion protein.

In some embodiments, the load solution in step i) comprises 2 to 3 M NaCl and has a pH of 6.0 to 8.0. In some embodiments, the elution solution in step iii) comprises 0.75-1.75 M NaCl and has a pH of 6.0 to 7.0. In some embodiments, the load solution in step iv) has a conductivity of 0.7-4.0 mS/cm and a pH of 7.0 and 9.0. In some embodiments, the load solution in step iv) has a conductivity of 0.7-4.0 mS/cm and a pH of 7.0 and 9.1. In some embodiments, the load solution in step vi) has a pH of 6.0 to 7.0 and a conductivity of 0.7 to 2.5 mS/cm. In some embodiments, the load solution in step vi) has a pH of 5.9 to 7.0 and a conductivity of 0.7 to 2.5 mS/cm.

In some embodiments, the elution solution in step viii) has a pH of 6.0 to 7.0 and a conductivity of 0.7 to 4.0 mS/cm. In some embodiments, the load solution in step i) comprises 0.25-3 M Na₂SO₄ or 0.25-0.6 M NH₄SO₄ and a pH of 5.5 to 6.5. 20. In some embodiments, the elution solution in step iii) comprises 0.3-0.5 M NH₄SO₄ and has a pH of 5.5 to 6.5.

In some embodiments, the hydrophobic interaction chromatography is selected from the group consisting of a POROS Benzyl ultra resin, a Hexyl-650 C resin, and a Phenyl-600M resin. In some embodiments, the hydrophobic interaction chromatography is a Phenyl-600M resin. In some embodiments, the anion exchange interaction chromatography is selected from the group consisting of a POROS 50HQ resin, a POROS XQ resin, and a Gigacap Q-650M resin. In some embodiments, the anion exchange interaction chromatography is a Gigacap Q-650M resin. In some embodiments, the cation exchange interaction chromatography is a strong cation exchanger. In some embodiments, the cation exchange interaction chromatography is a mixed mode resin. wherein the cation exchange interaction chromatography is selected from the group consisting of a Capto MMC resin, a CMM Hypercel resin, a Capto SP impres resin, a Fracto gel SO3-resin, a GigaCap S-650S resin, and a POROS XS resin. In some embodiments, the cation exchange interaction chromatography is a POROS XS resin.

In some embodiments, the biologically active domain is an asparaginase subunit. In some embodiments, the asparaginase is selected from the group consisting of an E. coli asparaginase and an Erwinia asparaginase. In some embodiments, the asparaginase subunit comprises the amino acid sequence set forth in SEQ ID NO:1, SEQ ID NO:5, or SEQ ID NO:7.

In some embodiments, the charge-shielded fusion protein comprises the amino acid sequence set forth in SEQ ID NO: 9 or SEQ ID NO:10

In some embodiments, the cell is a bacterial cell. In some embodiments, the cell is an E. coli cell or a Pseudomonas cell.

Also provided herein is a charge-shielded protein produced by the methods provided herein.

In some embodiments provided herein is composition comprising a charge-shielded protein and a pharmaceutically acceptable carrier.

In some embodiment, provided herein is a method of treatment comprising administering a composition comprising a charge-shielded protein or a pharmaceutical composition comprising a charge-shielded protein to an individual in need thereof.

Also provided herein is a composition comprising a PASylated asparaginase, wherein the PASylated asparaginase is at least 45% pure.

### BRIEF DESCRIPTION OF THE FIGURES

FIG. 1 shows an SDS-PAGE of eluates from a POROS HQ anion exchange column as an initial protein capture step. Arrow indicates band corresponding to PF745.

FIG. 2 shows an SDS-PAGE of eluates from a POROS XS cation exchange column as an initial protein capture step.

FIG. 3 shows an SDS-CGE image of fractions from representative Butyl-650M chromatography step. Equal volume (4 μL) of the load, flowthrough from load (FT), elution, and strip fractions were loaded onto SDS-CGE for purity analysis. Arrow indicates band corresponding to PF745.

FIG. 4 shows an overlay comparison of a representative POROS HQ chromatogram from three runs. Chromatograms display volume in mL on the X-axis, absorbance at 280 nm in mAU on the left Y-axis and conductivity in mS/cm on right Y-axis.

FIG. 5 shows an SDS-CGE image of fractions from a POROS XS chromatography step. Equal volume (4 μL) of the load, flowthrough from load (FT), wash, elution, strip 1, and strip 2 fractions were loaded onto CGE for purity analysis. Arrow indicates band corresponding to PF745.

FIG. 6 shows an SDS-CGE of flow-throughs from a high-hydrophobicity plate of HIC resins. Triplicate columns represent the flow-throughs from wells loaded with kosmotrope concentrations denoted by A, B, C, or D. “A” was 0.25 M sodium sulfate, “B” was 0.5 M ammonium sulfate, “C” was 2 M NaCl, and “D” was 3 M NaCl. The MW of the expected target band (PF745) is denoted with an arrow on the left side of the graphic.

FIG. 7 shows an SDS-CGE of elutions from a high-hydrophobicity plate of HIC resins. Triplicate columns represent the elutions from wells loaded with kosmotrope concentrations denoted by A, B, C, or D. “A” was 0.25 M sodium sulfate, “B” was 0.5 M ammonium sulfate, “C” was 2 M NaCl, and “D” was 3 M NaCl. The MW of the expected PF745 band is denoted with an arrow on the left side of the graphic.

FIG. 8 shows an SDS-CGE image of flow-throughs from a low-hydrophobicity plate of HIC resins.

FIG. 9 shows an SDS-CGE image of elutions from a low-hydrophobicity plate of HIC resins.

FIG. 10 shows an SDS-CGE image of Phenyl-600M and Benzylultra chromatography demonstrating enrichment of PF745 in fractions 1B2-1C4 for the Phenyl-600M and fractions 2A5-2C3 for Benzylultra.

FIG. 11 shows an SDS-CGE image from anion-exchange resin screening. The target (PF745) purity is displayed above the main band in each lane.

FIG. 12 shows SDS-CGE purity of flow-through fractions from POROS 50 HQ, POROS XQ, and GigaCap Q-650M chromatography runs.

FIG. 13 shows a representative chromatogram of AEX using GigaCap Q-650M.

FIG. 14 shows an SDS-CGE image of flow-through fractions (in triplicate lanes) from mixed-mode cation exchange resins. The top panel was run at pH 5.7 and the bottom panel was run at pH 6.0.

FIG. 15 shows an SDS-CGE image of Capto Core 400 load and flow-through fractions at various pH and salt concentrations as indicated above each lane. The % purity is shown above each lane.

FIG. 16 shows an SDS-CGE image of NH2-750F load and flow-through fractions as various pH and conductivities as indicated above each lane. The % purity is shown above each lane.

FIG. 17 shows an SDS-CGE image of CaPure-HA fractions: load, flow through (FT), wash and elution, with binding conditions indicated above each set of lanes.

FIG. 18 shows an SDS-CGE image of PPG-600M fractions: load, flow through (FT), wash and elution, with binding conditions indicated above each set of lanes.

### DETAILED DESCRIPTION

### I. Methods for Purifying Charge-Shielded Proteins

In some embodiments, the methods provided herein comprise purifying a charge-shielded protein using one or more chromatography steps; in some embodiments, the method comprises a hydrophobic interaction chromatography (HIC) as a first chromatography step. As used herein, the term “chromatography” comprises a method of separating a mixture (e.g., a mixture of proteins within a cell lysate or periplasmic releasate). In some embodiments, chromatography comprises separating a mixture, such as a cell lysate or periplasmic releasate, by passing it in a solution (e.g., load solution, mobile phase), through a medium which is on a fixed material (e.g., resin, stationary phase). A solution within a chromatography system may comprise as liquid (e.g., liquid chromatography) or vapor (e.g., gas chromatography). In some embodiments, chromatography separates a mixture in a solution through a medium which is on a fixed material, wherein the components of the mixture move at different rates causing them to separate from one another.

The composition of the specific load solution and/or resin may determine the rate at which the components of a mixture travel. For example, certain components of a mixture may travel more slowly through the resin (e.g., a longer retention time), while other components of the same mixture may travel more quickly through the resin (e.g., a shorter retention time), when a specific load solution and/or resin is used.

In some embodiments, chromatography separations of mixtures further comprises a resin (e.g., stationary phase), a load solution (e.g., buffer, mobile phase), and a column. The composition of the resin and buffer may be dependent on, and specific to, the particular chromatography method as described herein. In some embodiments, the chromatography column contains the resin, allowing the load solution comprising the mixture to be separated by chromatography, to pass through. In some embodiments, the column is a glass, borosilicate glass, acrylic glass, or stainless steel chromatography column.

In some embodiments, the methods provided herein relate to a capture purification step wherein a cell lysate or periplasmic releasate is applied to a hydrophobic interaction chromatography column A “cell lysate” as used herein comprises contents of a lysed cell. A “periplasmic releasate” as used herein comprises contents of a periplasm produced by lysis of an outer membrane. In some embodiments, a periplasmic releasate is a subfraction of a cell lysate. In some embodiments, a cell lysate or periplasmic releasate comprises a charge-shielded protein that has been expressed within the cell. A lysed cell may be obtained by breaking down the membrane of a cell, often by viral, enzymatic, or osmotic mechanisms, to disrupt the integrity of the cellular membrane. In some embodiments, a cell is lysed by physical disruption, including but not limited to, sonication, mechanical techniques (e.g., waring blender polytron), liquid homogenization (e.g., using a dounce homogenizer, Potter-Elvehjem homogenizer, microfluidizer, or a French press), freeze thaw, and manual grinding (e.g., mortar and pestle). In alterative embodiments, a cell is lysed by solution-based lysis, wherein the cell is contacted with a cell lysis buffer that breaks open the cells and releases intracellular contents. For example, a cell may be lysed using a solution of buffered salts (e.g., Tris-HCl or MES) and ionic salts (e.g., NaCl or KCl). In some embodiments, additional components including protease inhibitors and detergents, such as Triton X-100 or SDS, may be added to cell lysis buffers to prevent the degradation of proteins released from the cell. In some embodiments, any known technique in the art is used to produce a cell lysate or periplasmic releasate.

In some embodiments, a periplasmic releasate is produced by selectively disrupting a bacterial outer membrane. Methods for disrupting bacterial outer membranes are known in the art. (see Wurm et al. Engineering in Life Sciences 17:215-222 (2017)). For example, treatment with guanidine HCl and/or triton, cernitrate, benzalkonium chloride, glycerol ethers, chloroform, TRIS, 1% glycine, polyethylenimine, Urea and DTT, mild heat shot and TRIS, and osmotic shock can all be used. In some embodiments, the outer membrane is disrupted during cultivation. In some embodiments, the outer membrane is disrupted post harvesting.

In some embodiments, soluble fractions of a cell lysate or periplasmic releasate comprising a charge-shielded protein are separated from the insoluble fractions of a cell lysate or periplasmic releasate using centrifugation, following lysis of the cell or extracellular membrane and prior to a first chromatography capture step. In some embodiments, soluble fractions of a cell lysate or periplasmic releasate are separated from insoluble fractions of a cell lysate or periplasmic releasate by centrifugation. In some embodiments, a cell lysate or periplasmic releasate is centrifuged at up to, greater than, or about 3,000×g, about 3,500×g, about 4,000×g, about 4,500×g, about 5,000×g, about 5,500×g, about 6,000×g, about 6,500×g, about 7,000×g, about 8,000×g, about 9,000×g, about 10,000×g, r about 11,000×g or about 15,000×g. In some embodiments, a cell lysate or periplasmic releasate is centrifuged at about 8,000-20,000×g, about 5,000-6,000×g, about 8,000-15,000×g, about 18,000×g, or about 20,000×g. In some embodiments, the cell lysate or periplasmic releasate is centrifuged for up to, greater than, or about 5 min, about 6 min, about 7 min, about 8 min, about 9 min, about 10 min, about 11 min, about 12 min, about 13 min, about 14 min, about 15 min, about 20 min or about 30 min In some embodiments, the cell lysate or periplasmic releasate is centrifuged for about 5-30 minutes, about 5-20 min, about 8-12 min, about 10-20 min, or about 15-30 minutes. A centrifugation may be performed at up to, greater than, or about 0° C., about 1° C., about 2° C., about 3° C., about 4° C., about 5° C., about 6° C., about 7° C., about 8° C., about 9° C., or about 10° C. In some embodiments, centrifugation is performed at about 0-10° C., or about 2-8° C.

In some embodiments, after centrifugation, the cell lysate or periplasmic releasate is subject to one or more filtration or clarification steps prior to a first capture chromatography step. In some embodiments, the cell lysate or periplasmic releasate is subject to ultrafiltration. In some embodiments a 0.2, 0.3, 0.4, 0.45 or 0.5 μm filter is used. In some embodiments, the cell lysate or periplasmic releasate is subject to dialysis. In some embodiments, buffer exchange is performed such that the cell lysate or periplasmic releasate is in a buffer suitable for application to a first hydrophobic interaction chromatography column.

In some embodiments, the soluble fraction of a cell lysate or periplasmic releasate isolated by centrifugation, comprising a charge-shielded protein is applied to a capture step. As used herein, a “capture step” comprises a first chromatography step that binds the protein of interest (e.g., a charge-shielded protein) from the cell lysate. In some embodiments, a first chromatography capture step isolates the protein of interest from whole cell lysate cell contaminants, including but not limited to, proteases and glycosidases, in addition to non-target host cell proteins. In some embodiments, a first chromatography capture step concentrates a target protein and preserves the target protein activity. In some embodiments, a first chromatography capture step may be optimized to maximize the purification of a target protein from cell contaminants (e.g., non-target host cell proteins). In some embodiments, prior to a first chromatography capture step, a charge-shielded protein in a cell lysate is about 5%, about 6%, about 7%, about 8%, about 9%, or about 10%, pure. In some embodiments, prior to a first chromatography capture step, a charge-shielded fusion protein in a cell lysate is about 5-10%, about 6-8%, or about 7-9% pure. In some embodiments, prior to a first chromatography capture step, a charge-shielded fusion protein in a cell lysate is about 5-30%, about 10-30%, or about 15-20% pure.

In some embodiments, the soluble fraction of a periplasmic releasate is applied to a capture step. In some embodiments, chromatography step that binds the protein of interest (e.g., a charge-shielded protein) from the periplasmic releasate. In some embodiments, a first chromatography capture step concentrates a target protein and preserves the target protein activity. In some embodiments, a first chromatography capture step may be optimized to maximize the purification of a target protein from cell contaminants (e.g., non-target host cell proteins) present in a periplasmic releasate. In some embodiments, prior to a first chromatography capture step, a charge-shielded protein in a cell periplasmic releasate is about 5%, about 6%, about 7%, about 8%, about 9%, about 15%, about 18%, about 20%, about 25% or about 30%, pure. pure. In some embodiments, prior to a first chromatography capture step, a charge-shielded fusion protein in a periplasmic releasate is about 5-30%, about 10-30%, or about 15-20% pure.

A method described herein may comprise using chromatography to purify a charge-shielded fusion protein (e.g., from a cell lysate or periplasmic releasate). In some embodiments, a method described herein may comprise using multiple chromatography steps to purify a charge-shielded fusion protein. In some embodiments, a method for purifying a charge-shielded fusion protein comprises one, two, three, four, five, six, or seven chromatography steps. In some embodiments, a method for purifying a charge-shielded fusion protein comprises 1-7, or 1-3, or 3-5 chromatography steps. Chromatography may comprise liquid chromatography or gas chromatography. In some embodiments, the method comprises HIC, anion exchange (AEX) chromatography, cation exchange (CEX) chromatography, ion exchange (IEX) chromatography, partition chromatography, normal-phase chromatography, displacement chromatography, reversed-phase chromatography (RPC), bioaffinity chromatography, aqueous normal-phase chromatography, high-performance liquid chromatography, flash chromatography, or other chromatography methods.

In some embodiments, a charge-shielded fusion protein has a purity of about 40%, about 50%, about 60%, about 70%, 80%, about 85%, about 90%, or about 95%, following a first chromatography capture step. In some embodiments, a charge-shielded fusion protein has a purity of about 50%-80%, or about 60%-80%, following a first chromatography capture step. In some embodiments, a charge-shielded fusion protein has a purity of at least 45% following a first chromatography capture step. In some embodiments, the purity of the charge-shielded protein is higher following a first HIC chromatography step compared to the purity of the charge-shielded protein using an ion exchange chromatography step. In some embodiments, the purity of the charge-shielded protein is higher following a first HIC chromatography step than the purity of the charge-shielded protein when purified according to the method of the biologically active domain.

A charge-shielded fusion protein may have increased purity compared to a single chromatography step, when a first chromatography step is combined with a second chromatography step. A charge-shielded fusion protein may have increased purity compared to a single chromatography step, when a first chromatography step is combined with a second and third chromatography step. A charge-shielded fusion protein may have increased purity compared to two chromatography steps, when first and second chromatography steps are combined with a third chromatography step.

In some embodiments, the method comprises a first chromatography, or capture, step (e.g., HIC). In some embodiments, a first HIC step is followed by a second HIC step. In some embodiments, a first HIC step is followed by an AEX chromatography step. Alternatively, a first HIC step may be followed by a CEX chromatography step. In some embodiments, a first HIC step is followed by a chromatography step comprised of any chromatography technique described herein, or otherwise known to one of ordinary skill in the art. The second chromatography step, following a first HIC step, is optionally followed by a third chromatography step. In one aspect, a third chromatography step is a CEX chromatography step. In another aspect, a third chromatography step is an AEX chromatography step. In some embodiments, a CEX chromatography step is performed after a first HIC step, and an AEX chromatography step. In some embodiments, an AEX chromatography step is performed after a first HIC step, and a CEX chromatography step. In alternative methods, a third chromatography step is comprised of any chromatography technique described herein, or otherwise known to one of ordinary skill in the art, and is performed after a first HIC step, and an AEX step. Further embodiments include a third chromatography step comprised of any chromatography technique described herein, or otherwise known to one of ordinary skill in the art, performed after a first HIC step, and a CEX step.

Between each chromatography step, one or more optional ultrafiltration (UF) and/or diafiltration (DF) (e.g., UF/DF) steps may be performed. In some embodiments, UF/DF is performed for concentration and buffer exchange between chromatography steps. For example, UF/DF may comprise separation by filtration. In some embodiments, an eluate from a chromatography step is contacted with a membrane under applied pressure. In some embodiments, this applied pressure drives the migration of the elution solution, buffer salts, and smaller non-target solution components, through the membrane. In some embodiments, the membrane retains the larger molecules (e.g., target proteins).

In some embodiments, the methods provided herein comprise using HIC as a first chromatography step. In some embodiments, HIC comprises a method for separating mixtures based on their hydrophobicity. HIC may comprise applying a mixture comprising a buffer and proteins, comprising both hydrophilic and hydrophobic regions, to an HIC resin within a chromatography column. In some embodiments, HIC specific resins are used to perform HIC as a first chromatography step. In some embodiments, HIC resins are high hydrophobicity HIC resins. In some embodiments, HIC resins are low hydrophobicity resins. In some embodiments, the purity of the composition comprising a charge-shielded fusion protein is about 40%, about 50%, about 60%, about 70%, about 80% about 85%, about 90%, or about 95%, following an HIC capture chromatography step. In some embodiments, a charge-shielded fusion protein has a purity of about 50%-80%, or about 60%-80% or 80%-95%, following an HIC capture chromatography step. In some embodiments, a charge-shielded fusion protein has a purity of at least 45% following an HIC capture chromatography step.

In some embodiments, an HIC resin has a pore size of up to, greater than, or about 500 Å, about 550 Å, about 600 Å, about 650 Å, about 700 Å, about 750 Å, about 800 Å, about 850 Å, about 900 Å, about 950 Å, about 1,000 Å, about 1,500 Å, or about 2,000 Å. In some embodiments, an HIC resin has a pore size between about 500-2,000 Å, about 700-1,000 Å, about 700-800 Å, or about 900-1,500 Å. In some embodiments, an HIC resin has a particle size of up to, greater than, or about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, or about 120 μm. In some embodiments, an HIC resin has a particle size between about 40-120 μm, about 60-100 μm, about 70-110 μm, and about 40-50 nm.

In some embodiments, an HIC resin is comprised of a matrix support base material, wherein the base material is a hydrophilic carbohydrate. An HIC resin base material may be cross-linked agarose or synthetic copolymer materials. In some embodiments, an HIC resin is comprised of a cross-linked polystyrene-divinylbenzenel base material or a hydroxylated methacrylate polymer base material. In some embodiments, an HIC resin is further comprised of a ligand functional group bound to the base material, wherein the ligand functional group is hydrophobic. An HIC ligand functional group may be a straight chain alkyl ligand demonstrating hydrophobicity, or an aryl ligand demonstrating mixed mode behavior, where both aromatic and hydrophobic interactions are possible. In some embodiments, the ligand functional group is an aromatic hydrophobic benzyl ligand, a phenyl ligand, or a C6 (hexyl) group. In some embodiments, an HIC resin is comprised of a cross-linked polylstyrene-divinylbenzenel base material bonded with an aromatic hydrophobic benzyl ligand functional group. In some embodiments, an HIC resin is comprised of a hydroxylated methacrylate polymer base material bonded with C6 (hexyl) groups. In some embodiments, an HIC resin is comprised of a hydroxylated methacrylate polymer base material bonded with phenyl functional groups. In some embodiments, the HIC resin is a POROS Benzyl ultra resin, a POROS Benzyl resin, a Capto Phenyl (high sub) resin, a Butyl-650M resin, a Hexyl-650C resin, a Phenyl-600M resin, a Capto Phenyl ImpRes resin, a Phenyl Sepharose HP resin, an Octyl Sepharose 4 FF resin, a Capto Octyl resin, a PPG-600M resin, or a POROS Ethyl resin.

Often, an HIC resin may be equilibrated using an equilibration buffer prior to applying a load solution comprising a charge-shielded fusion protein. In some embodiments, the HIC equilibration buffer comprises a buffered salt solution. In some embodiments, the HIC equilibration buffer comprises Tris, EDTA, and a salt (e.g., NaCl). In some embodiments, the HIC equilibration buffer is equilibrated to a pH of about 5.0-10.0, or up to, greater than, or about pH 5.0, about pH 6.0, about pH 7.0, about pH 8.0, about pH 9.0, or about pH 10.0. In some embodiments, the HIC equilibration buffer is selected based on the specific HIC resin use for a first chromatography capture step. Optionally, an HIC equilibration solution comprises additives, including but not limited to, detergents, alcohols, and chaotropic salts.

In some embodiments, the charge-shielded fusion protein is applied to an HIC resin in a mixture, wherein the mixture comprising the charge-shielded fusion protein comprises a load solution. In some embodiments, the load solution comprising the charge-shielded fusion protein is applied to an HIC resin. In some embodiments, the load solution comprises a salt solution. In some embodiments, the salt solution of the HIC load solution comprises NaCl, (NH₄)₂SO₄, Na₂SO₄, KCl, or CH₃COONH₄. In some embodiments, the salt solution of the HIC load solution comprises about 1 M NaCl, about 2 M NaCl, about 3 M NaCl, about 4 M NaCl, or about 5 M NaCl. In some embodiments, the salt solution comprises between about 1-5 M NaCl, or between about 2-3 NaCl. In some embodiments, the HIC load solution comprising the charge-shielded fusion protein added to an HIC resin has a pH of no more than, greater than, or about 5.0, about 5.5, about 6.0, about 6.5, about 7.0, about 7.5, about 8.0, about 8.5, or about 9.0. In some embodiments, the HIC load solution comprising the charge-shielded fusion protein added to an HIC resin has a pH of about 5.0-9.0, or a pH of about 6.0-8.0. Optionally, a load solution comprises additives, including but not limited to, detergents, alcohols, and chaotropic salts.

In some embodiments, the load solution comprises about 0.25 to about 3 M Na₂SO₄, such as about 0.4 to about 3.0 M Na₂SO₄, about 0.5 to about 3 M Na₂SO_{4,} about 0.4 to about 2 M Na₂SO₄, or about 0.4 to about 1.0 M Na₂SO₄. In some embodiments, the load solution comprises about 0.6 M Na₂SO₄. In some embodiments, the load solution has a pH of 5.5 to 6.5, such as pH 5.5 to 6.3, pH 5.6 to 6.3, or pH 5.7 to 6.2. In some embodiments, the load solution has a pH of about pH 5.9. In some embodiments, the load solution comprises about 0.25 to about 0.6 M NH₄SO₄, about 0.3 to about 0.6 M NH₄SO₄, or about 0.4 to about 0.6 M NH₄SO₄.

One or more wash steps may be performed using a wash buffer, following the applying the HIC loading solution comprising the charge-shielded fusion protein to the HIC resin. A wash buffer is selected based on the HIC load solution and the specific HIC resin, and it will be obvious to those skilled in the art that various wash buffers can be used. In some embodiments, a wash buffer comprises a salt solution. In some embodiments, the wash buffer comprises NaCl, (NH₄)₂SO₄, Na₂SO₄, KCl, or CH₃COONH₄. In some embodiments, the wash buffer further comprises Tris and EDTA. Optionally, a wash buffer comprises additives, including but not limited to, detergents, alcohols, and chaotropic salts. In some embodiments, the HIC wash buffer is the same as the HIC equilibration buffer. Alternatively, the HIC wash buffer may be different than the HIC equilibration buffer.

In some embodiments, the purified charge-shielded fusion protein is eluted from the HIC resin, optionally following one or more washes. The HIC elution solution comprises a salt solution. In some embodiments, the HIC elution solution salt solution is an NaCl buffer. In some embodiments, the NaCl buffer comprises about 0.6 M NaCl, about 0.65 M NaCl, about 0.7 M NaCl, about 0.75 M NaCl, about 0.8 M NaCl, about 0.85 M NaCl, about 0.9 M NaCl, about 1 M NaCl, about 1.2 M NaCl, about 1.5 M NaCl, about 1.75 M NaCl, about 2 M NaCl, or about 2.5 M NaCl. In some embodiments, the NaCl buffer comprises about 0.6-2.5 M NaCl or about 0.75-1.75 M NaCl. In some embodiments, the HIC elution solution has a pH of about 5.5, about 6.0, about 6.5, about 7.0, or about 7.5. In some embodiments, the HIC elution solution has a pH of about 5.5-7.5, or a pH of about 6.0-7.0. Optionally, an elution solution comprises additives, including but not limited to, detergents, alcohols, and chaotropic salts.

In some embodiments, the HIC elution solution comprises about 0.3 to about 0.5 M NH4SO4 and has a pH of about 5.5 to about 6.5. In some embodiments, the HIC elution solution comprises about 0.35 to about 0.45 M NH₄SO₄ or about 0.4 M NH₄SO₄. In some embodiments, the HIC elution solution has a pH of about pH 5.6 to about 6.4, about pH 5.7 to about 6.2, or about pH 5.9.

In some embodiments, HIC is performed at about room temperature. In some embodiments, HIC is performed at about 15° C. to about 28° C., or about 18° C. to about 25° C.

In some embodiments, HIC is performed 1, 2, 3, 4, 5, 6, 7, 8, 9, 10, 11, 12, 13, 14, or 15 times. In some embodiments, a first HIC capture step is performed 1-15 times, 3-6 times, 8-10 times, or 9-15 times. Optionally, an eluate from a first HIC capture step may be stored at 0° C., about 1° C., about 2° C., about 3° C., about 4° C., about 5° C., about 6° C., about 7° C., about 8° C., about 9° C., or about 10° C., until ready for further processing. In some embodiments, an eluate from a first HIC capture step is stored at about 4° C. to about 8° C. In some embodiments, an eluate from a first HIC capture step is stored at about 5-25° C., about 2-8° C., about 10 -20° C., or about 18° C.-25° C., until ready for further processing. In some embodiments, the eluate is stored for about up to 8 hours at about 25° C. In some embodiments, the eluate is stored for greater than 24 hours at about 4° C. to about 8° C.

In some embodiments, the methods provided herein comprise purifying a charge-shielded fusion protein using one or more chromatography steps, and in some embodiments, the method comprises an AEX chromatography as a chromatography step following HIC. In some embodiments, the AEX chromatography step is a second chromatography step, subsequent to the first HIC step. AEX chromatography is a process that separates substances based on their net surface charge, using an IEX resin containing positively charged groups. In solution, the resin is coated with positively charged counter-ions. Therefore, the positively charged groups on an AEX resin will bind negatively charged proteins in solutions. In some embodiments, the AEX resin used in the methods described herein is a strong anion exchange resin. In some embodiments, the AEX resin used in the methods described herein is a weak anion exchange resin. The classification of an AEX resin as a “strong” or “weak” anion exchanger refers to the extent that the ionization state of the resin functional groups vary with pH. For example, a weak AEX resin is ionized over a limited pH range (e.g., functional groups take up or lose protons with changes in buffer pH), while a strong AEX resin shows no variation in ion exchange capacity with changes in pH (e.g., functional group do not vary and remain fully charged over a broad pH range).

Often, an AEX resin may be equilibrated using an equilibration buffer prior to applying an AEX load solution comprising a charge-shielded fusion protein. In some embodiments, the AEX equilibration buffer comprises a buffered salt solution. In some embodiments, the AEX equilibration buffer comprises Tris, EDTA, and a salt (e.g., NaCl). In some embodiments, the AEX equilibration buffer is equilibrated to a pH of about 5.0-10.0, or up to, greater than, or about pH 5.0, about pH 6.0, about pH 7.0, about pH 8.0, about pH 9.0, or about pH 10.0. In some embodiments, the AEX equilibration buffer is selected based on the specific AEX resin use for a second chromatography step. Optionally, an AEX equilibration solution comprises additives, including but not limited to, detergents, alcohols, and chaotropic salts.

In some embodiments, an AEX resin has a pore size of up to, greater than, or about 500 Å, about 600 Å, about 700 Å, about 800 Å, about 900 Å, about 1,000 Å, about 2,000 Å, about 3,000 Å, about 4,000 Å, about 5,000 Å, about 6,000 Å, about 7,000 Å, about 8,000 Å, about 9,000 Å, or about 10,000 Å. In some embodiments, an AEX resin has a pore size of about 500-10,000 Å, about 500-800 Å, about 900-1,200 Å, or about 5,000-10,000 Å. In some embodiments, an AEX resin has a particle size of up to, greater than, or about 50 μm, about 55 μm, about 60 μm, about 65 μm, about 70 μm, about 75 μm, about 80 μm, about 85 μm, about 90 μm, about 95 μm, or about 100 μm. In some embodiments, an AEX resin has a particle size of about 50-100 μm, about 70-80 μm, about 50-90 μm, or about 80-100 μm.

In some embodiments, an AEX resin is comprised of a poly[styrene-divinylbenzene] or hydroxylated methacrylic polymer base material. An AEX resin base material may optionally be coated with an additional polyhydroxyl surface coating, to ensure low non-specific binding. In some embodiments, an AEX resin is further comprised of a ligand functional group bound to the base material, wherein the ligand functional group is positively charged, or basic. An AEX ligand functional group may be a weak or strong anion exchanger. For example, a weak AEX ligand functional group may comprise diethylaminoethyl or diethylaminopropyl. Alternatively, a strong AEX ligand functional group may comprise a quaternary ammonium or amine group. In some embodiments, an AEX resin is comprised of a rigid, highly porous, crosslinked poly[styrene-divinylbenzene] base material with an additional polyhydroxyl surface coating to ensure low nonspecific binding, bonded with quaternized polyethyleneimine functional groups. In some embodiments, an AEX resin is comprised of a rigid, highly porous, crosslinked polystyrene -divinylbenzenel base material with an additional polyhydroxyl surface coating to ensure low nonspecific binding, bonded with a fully quaternized quaternary amine In some embodiments, an AEX resin is comprised of a hydroxylated methacrylic polymer base material that has been chemically modified to provide a higher number of anionic binding sites, and bonded with quaternary amine strong AEX functional groups. In some embodiments, an AEX resin is a POROS 50HQ resin, a POROS XQ resin, a Gigacap Q-650M resin, a Super Q-650M resin, or a NH2-750F resin.

In some embodiments, the charge-shielded fusion protein is applied to an AEX resin in a mixture, wherein the mixture comprising the charge-shielded fusion protein comprises a load solution, that comprised of the eluate from the HIC step. In some embodiments, the load solution comprising the charge-shielded fusion protein is applied to an AEX resin. In some embodiments, the load solution comprises a salt. In some embodiments, the AEX load solution has a conductivity of no more than, greater than, or about 0.5 mS/cm, about 0.6 mS/cm, about 0.7 mS/cm, about 0.8 mS/cm, about 0.9 mS/cm, about 1.0 mS/cm, about 2.0 mS/cm, about 3.0 mS/cm, about 4.0 mS/cm, about 5.0 mS/cm, and about 6.0 mS/cm. In some embodiments, the AEX load solution has a conductivity of about 0.5-6.0 mS/cm, or a conductivity of about 0.7-4.0 mS/cm. In some embodiments, the AEX load solution has a pH of no more than, greater than, or about 6.0, about 6.5, about 7.0, about 7.5, about 8.0, about 8.5, about 9.0, about 9.5, or about 10.0. In some embodiments, the AEX load solution has a pH of about 6.0-10.0, or a pH of about 7.0-9.0. In some embodiments, the AEX load solution has a pH of 7.0 to 9.1.

One or more wash steps may be performed using a wash buffer, following the applying the AEX loading solution comprising the charge-shielded fusion protein to the AEX resin. A wash buffer is selected based on the AEX load solution and the specific AEX resin. In some embodiments, a wash buffer comprises a salt solution. In some embodiments, the wash buffer comprises NaCl, (NH₄)₂SO₄, Na₂SO₄, KCl, or CH₃COONH₄. In some embodiments, the wash buffer further comprises Tris and EDTA. Optionally, a wash buffer comprises additives, including but not limited to, detergents, alcohols, and chaotropic salts. In some embodiments, the AEX wash buffer is the same as the AEX equilibration buffer. Alternatively, the AEX wash buffer may be different than the AEX equilibration buffer.

In some embodiments, the purified charge-shielded fusion protein is applied to the AEX resin and the flowthrough is collected, optionally following one or more washes. In some embodiments, all AEX flowthrough and all washes are collected. A second chromatography step, optionally comprising AEX, may be performed one or more times in order to obtain sufficient material for subsequent downstream processing. In some embodiments, an AEX chromatography step is performed 1, 2, 3, 4, 5, 6, 7, 8, 9, 10, 11, 12, 13, 14, or 15 times. In some embodiments, an AEX chromatography step is performed 1-15 times, 3-6 times, 8-10 times, or 9-15 times. Optionally, the flowthrough from an AEX chromatography step may be stored at 0° C., about 1° C., about 2° C., about 3° C., about 4° C., about 5° C., about 6° C., about 7° C., about 8° C., about 9° C., or about 10° C., until ready for further processing. In some embodiments, an eluate from an AEX chromatography step is stored at about 0-10° C., or about 2-8° C., until ready for further processing. In some embodiments, an eluate from an AEX chromatography step is stored at about 4° C. to about 8° C. In some embodiments, an eluate from a AEX chromatography step is stored at about 5-25° C., about 2-8° C., about 10-20° C., or about 18° C.-25° C., until ready for further processing. In some embodiments, the eluate is stored for about up to 8 hours at about 25° C. In some embodiments, the eluate is stored for greater than 24 hours at about 4° C. to about 8° C.

In some embodiments, the methods provided herein comprise purifying a charge-shielded fusion protein using one or more chromatography steps, and in some embodiments, the method comprises an CEX chromatography as a chromatography step following HIC. In some embodiments, the CEX chromatography step is a second chromatography step, subsequent to the first HIC step. In some embodiments, the CEX chromatography step is a third chromatography step, subsequent to the first HIC step and AEX, chromatography step. CEX chromatography is a process that separates substances based on their net surface charge, using an IEX resin containing negatively charged groups. In solution, the resin is coated with negatively charged counter-ions. Therefore, the negatively charged groups on a CEX resin will bind positively charged proteins in solutions. In some embodiments, the CEX resin used in the methods described herein is a strong cation exchange resin. In some embodiments, the CEX resin used in the methods described herein is a weak cation exchange resin. The classification of an CEX resin as a “strong” or “weak” anion exchanger refers to the extent that the ionization state of the resin functional groups vary with pH. For example, a weak CEX resin is ionized over a limited pH range (e.g., functional groups take up or lose protons with changes in buffer pH), while a strong CEX resin shows no variation in ion exchange capacity with changes in pH (e.g., functional group do not vary and remain fully charged over a broad pH range).

In some embodiments, the CEX resin is a mixed mode resin. Mixed mode chromatography comprises chromatography methods that utilize more than one form of interaction between the stationary phase and load solution in order to achieve separation of the target protein. Most mixed mode phases are typically bonded silica or polymeric reversed phase based materials bonded with an ion-exchange ligand functional group. For example, a mixed mode CEX resin may comprise a negatively charged sulfonate group covalently bonded to the reversed phase backbone.

Often, a CEX resin may be equilibrated using an equilibration buffer prior to applying a CEX load solution comprising a charge-shielded fusion protein. In some embodiments, the CEX equilibration buffer comprises a buffered salt solution. In some embodiments, the CEX equilibration buffer comprises Tris, EDTA, and a salt (e.g., NaCl). In some embodiments, the CEX equilibration buffer is equilibrated to a pH of about 5.0-10.0, or up to, greater than, or about pH 5.0, about pH 6.0, about pH 7.0, about pH 8.0, about pH 9.0, or about pH 10.0. In some embodiments, the equilibration buffer is selected based on the specific CEX resin use for a second chromatography step. Optionally, an equilibration solution comprises additives, including but not limited to, detergents, alcohols, and chaotropic salts.

In some embodiments, a CEX resin has a pore size of up to, greater than, or about 500 Å, about 600 Å, about 700 Å, about 800 Å, about 900 Å, about 1,000 Å, or about 2,000 Å. In some embodiments, a CEX resin has a pore size of about 500-2,000 Å, about 800-1,000 Å, or about 700-900 Å. In some embodiments, a CEX resin has a particle size of up to, greater than, or about 10 μm, about 20 μm, about 30 μm, about 40 μm, about 50 μm, about 60 μm, about 70 μm, about 80 μm, about 90 μm, or about 100 μm. In some embodiments, a CEX resin has a particle size of about 20-100 μm, about 30-50 μm, about 50-80 μm, or about 80-100 μm.

In some embodiments, a CEX resin is comprised of a polystyrene-divinylbenzenel, methacrylate polymer, agarose, or cellulose base material. A CEX resin base material may be coated with an additional polyhydroxyl surface coating to ensure low nonspecific binding. In some embodiments, a CEX resin is further comprised of a ligand functional group bound to the base material, wherein the ligand functional group is negatively charged, or acidic. A CEX ligand functional group may be a weak or strong cation exchanger. For example, a weak CEX ligand functional group may comprise a carboxymethyl group. Alternatively, a strong CEX ligand functional group may comprise sulfonic acids (e.g., methyl sulfonate, sulfonyl, sulfoisobutyl, sulphopropyl), carboxylic acid (e.g., carboxymethyl), or phosphonic acids. In some embodiments, a CEX ligand functional group may comprise multimodal (e.g., mixed mode) functional groups, including primary amines, or groups providing hydrogen bonding and hydrophobic interaction sites, in addition to the negatively charged CEX groups.

In some embodiments, a CEX resin is comprised of a rigid, highly porous, crosslinked polystyrene-divinylbenzenel base material with an additional polyhydroxyl surface coating to ensure low nonspecific binding, bonded with a high density of negatively charged sulphopropyl functional groups. In some embodiments, a CEX resin is comprised of a rigid, high-flow agarose base matrix bonded with a multimodal weak CEX ligand functional group, containing a carboxylic group and additional groups providing hydrogen bonding and hydrophobic interaction sites. In some embodiments, a CEX resin is comprised of a rigid cellulose base matrix bonded with a ligand, containing both a primary amine and a carboxyl group, that confers CEX and hydrophobicity properties. In some embodiments, a CEX resin is comprised of a high-flow agarose base matrix bonded with a negatively charged sulfonate (SP) group. In some embodiments, a CEX resin is comprised of a synthetic methacrylate polymer base material bonded with negatively charged sulfoisobutyl functional ion exchanger groups, via linear polymer chains. In some embodiments, a CEX resin is comprised of a high resolution, high capacity CEX resin comprising a methacrylate polymer base material chemically modified to provide a higher number of cationic binding sites, bonded with sulfopropyl (S) strong CEX functional groups. In some embodiments, a CEX resin is a Capto MMC resin, a CMM Hypercel resin, a Capto SP impres resin, a Fracto gel SO3—resin, a GigaCap S-650S resin, a POROS XS resin, a MX-TRP-650M resin, a Sulfate-650F resin, a NH2-750F resin, a CaPure-HA resin, or a PPG-600M resin.

In some embodiments, the charge-shielded fusion protein is applied to a CEX resin in a mixture, wherein the mixture comprising the charge-shielded fusion protein comprises a load solution, that comprised of the elution from the previous chromatography step (e.g., AEX chromatography or HIC). In some embodiments, the load solution comprising the charge-shielded fusion protein is added to a CEX resin, and comprises about a salt solution. In some embodiments, the CEX load solution has a conductivity of no more than, greater than, or about 0.5 mS/cm, about 0.6 mS/cm, about 0.7 mS/cm, about 0.8 mS/cm, about 0.9 mS/cm, about 1.0 mS/cm, about 2.0 mS/cm, about 2.5 mS/cm, about 3.0 mS/cm, about 3.5 mS/cm, and about 4.0 mS/cm. In some embodiments, the CEX load solution has a conductivity of about 0.5-4.0 mS/cm, or a conductivity of about 0.7-2.5 mS/cm. In some embodiments, the CEX load solution has a pH of no more than, greater than, or about 5.0, about 5.5, about 6.0, about 6.5, about 7.0, about 7.5, or about 8.0. In some embodiments, the CEX load solution has a pH of about 5.0-8.0, or a pH of about 6.0-7.0. In some embodiments, the CEX load solution has a pH of 5.9 to 7.0.

One or more wash steps may be performed using a wash buffer, following the applying the CEX loading solution comprising the charge-shielded fusion protein to the CEX resin. A wash buffer is selected based on the CEX load solution and the specific CEX resin, and it will be obvious to those skilled in the art that various wash buffers can be used. In some embodiments, a wash buffer comprises a salt solution. In some embodiments, the wash buffer comprises NaCl, (NH₄)₂SO₄, Na₂SO₄, KCl, or CH₃COONH₄. In some embodiments, the wash buffer further comprises Tris or MES and EDTA. Optionally, a wash buffer comprises additives, including but not limited to, detergents, alcohols, and chaotropic salts. In some embodiments, the CEX wash buffer is the same as the CEX equilibration buffer. Alternatively, the CEX wash buffer may be different than the CEX equilibration buffer.

In some embodiments, the purified charge-shielded fusion protein is eluted from the CEX resin, optionally following one or more washes. The CEX elution solution comprises a salt solution. In some embodiments, the CEX elution solution has a conductivity of no more than, greater than, or about 0.5 mS/cm, about 0.6 mS/cm, about 0.7 mS/cm, about 0.8 mS/cm, about 0.9 mS/cm, about 1.0 mS/cm, about 2.0 mS/cm, about 2.5 mS/cm, about 3.0 mS/cm, about 3 mS/cm, about 4.0 mS/cm, or about 5.0 mS/cm. In some embodiments, the CEX elution solution has a conductivity of about 0.5-5.0 mS/cm, about 0.7-4.0 mS/cm, about 1.0-2.0 mS/cm, or about 3.0-4.0 mS/cm. In some embodiments, the CEX elution solution has a pH of about 5.5, about 6.0, about 6.5, about 7.0, or about 7.5. In some embodiments, the CEX elution solution has a pH of about 5.5-7.5, or a pH of about 6.0-7.0.

A third chromatography step, optionally comprising CEX, may be performed one or more times in order to obtain sufficient material for subsequent downstream processing. In some embodiments, a CEX chromatography step is performed 1, 2, 3, 4, 5, 6, 7, 8, 9, 10, 11, 12, 13, 14, or 15 times. In some embodiments, a CEX chromatography step is performed 1-15 times, 3-6 times, 8-10 times, or 9-15 times. Optionally, an eluate from a CEX chromatography step may be stored at 0° C., about 1° C., about 2° C., about 3° C., about 4° C., about 5° C., about 6° C., about 7° C., about 8° C., about 9° C., or about 10° C., until ready for further processing. In some embodiments, an eluate from an AEX chromatography step is stored at about 0-10° C., or about 2-8° C., until ready for further processing.

### II. Methods of Producing a Charge-Shielded Fusion Protein

In some embodiments, the methods provided herein comprise culturing a cell comprising nucleic acid encoding the charge-shielded protein to produce a charge-shielded fusion protein and purifying the charge-shielded fusion protein. Host cells for the expression of polypeptides are well known in the art and comprise prokaryotic cells as well as eukaryotic cells, e.g. E. coli cells, Pseudomonas fluorescens cells, yeast cells, invertebrate cells, CHO-cells, CHO-K1-cells, Hela cells, COS-1 monkey cells, melanoma cells such as Bowes cells, mouse L-929 cells, 3T3 lines derived from Swiss, Balb-c or NIH mice, BHK or HaK hamster cell lines.

In some embodiments, nucleic acid encoding the charge-shielded protein is in a vector. In some embodiments, nucleic acid encoding the charge-shielded protein is integrated into the host cell chromosome.

Preferably, said vector is an expression vector and/or a gene transfer or targeting vector. Expression vectors derived from viruses such as retroviruses, vaccinia virus, adeno-associated virus, herpes viruses or bovine papilloma virus may be used for delivery of the polynucleotides or vector of the invention into targeted cell populations. The vectors containing the nucleic acid molecules of the invention can be transferred into the host cell by well-known methods, which vary depending on the type of cellular host.

The charge-shielded fusion protein may be produced by recombinant DNA technology, e.g. by cultivating a cell comprising the described nucleic acid molecule or vectors which encode the charge-shielded fusion protein and isolating said biologically active protein from the culture. The charge-shielded fusion protein may be produced in any suitable cell-culture system including prokaryotic cells, e.g. E. coli (e.g. BL21, W3110, or JM83), P. fluorescens, or Bacillus subtilus; or eukaryotic cells, e.g. Pichia pastoris yeast strain X-33 or CHO cells. Further suitable cell lines known in the art are obtainable from cell line depositories, like the American Type Culture Collection (ATCC). The term “prokaryotic” is meant to include bacterial cells while the term “eukaryotic” is meant to include yeast, higher plant, insect and mammalian cells. The transformed hosts can be grown in fermenters and cultured according to techniques known in the art to achieve optimal cell growth. In a further embodiment, the present invention relates to a process for the preparation of a biologically active protein described above comprising cultivating a cell of the invention under conditions suitable for the expression of the biologically active protein and isolating the biologically active protein from the cell or the culture medium.

Further examples of methods, vectors, and translation and transcription elements, and other elements useful in the methods herein are described in, e.g.: U.S. Pat. No. 5,055,294 to Gilroy and U.S. Pat. No. 5,128,130 to Gilroy et al.; U.S. Pat. No. 5,281,532 to Rammler et al.; U.S. Pat. Nos. 4,695,455 and 4,861,595 to Barnes et al.; U.S. Pat. No. 4,755,465 to Gray et al.; and U.S. Pat. No. 5,169,760 to Wilcox.

### III. Charge-Shielded Fusion Proteins

In some embodiments, the charge-shielding domain is located at the N-terminus of the fusion protein. In some embodiments, the charge-shielding domain is located at the C-terminus of the fusion protein. In some embodiments, the charge-shielding domain is located N-terminal to the biologically active domain. In some embodiments, the charge-shielding domain is located C-terminal to the biologically active domain. In some embodiments, the charge-shielded fusion protein comprises a peptide linker between the charge-shielding domain and the biologically active domain.

In some embodiments, the fusion proteins provided herein comprise a biologically active domain and a charge-shielding domain. In some embodiments, the charge shielding domain prevents or reduces binding of the biologically active domain to an ion exchange chromatography resin. In some embodiments, the charge-shielding domain increases the hydrophobicity of the fusion protein. In some embodiments, the charge shielding domain covers charged regions of the biologically active domain.

“Biologically active domain” as used herein is a protein or peptide that by itself, or in association with another molecule (such as a protein, lipid, nucleic acid, or other monomer(s)), has a biological activity. For example, a “biologically active domain” includes a subunit of a multimeric protein complex.

In some embodiments, the charge shielding domain is uncharged. In some embodiments, the charge shielding domain has a pI of about 7, such as about 6.5 to about 7.5, about 6.6 to about 7.4, about 6.7 to about 7.3, about 6.8 to about 7.2 or about 6.9 to about 7.1. In some embodiments, the charge shielding domain has a pI of 5 to 9, 5 to 6, 5 to 7, 7 to 8, or 7 to 9. In some embodiments, the charge shielding domain comprises uncharged amino acids. In some embodiments, the charge-shielding domain comprises polar amino acids. In some embodiments, the charge-shielding domain comprises non polar amino acids. In some embodiments, the charge-shielding domain consists of proline, alanine and serine. In some embodiments, the charge-shielding domain consists of proline and alanine.

In some embodiments, the charge-shielding domain has a molecular weight of from 10 to 200 kDa, such as from 10 to 100 kDa, 10 to 80 kDa, 10 to 60 kDa, or 10 to 40 kDa. In some embodiments, the charge-shielding domain has a molecular weight from 10 to 20 kDa.

In some embodiments, the charge-shielded fusion protein forms a multimeric protein, In some embodiments, the charge-shielded protein forms a dimer, trimer, tetramer, hexamer or octamer. In some embodiments, the charge-shielded fusion protein forms a tetramer.

In some embodiments, the molecular weight of the multimeric (such as tetrameric) charge-shielded protein is between 50 to 500 kDa, 75 to 300 kDa, or 100 to 250 kDa.

In some embodiments, the molecular weight of the charge-shielding domain is less than that of the biologically-active domain. In some embodiments, the molecular weight of the charge-shielding domain is less than 80%, less than 70%, less than 60%, less than 50% less than 40%, less than 30%, or less than 20% of the molecular weight of the biologically active domain.

In some embodiments, the molecular weight of the charge-shielding domain is greater than that of the biologically active domain. In some embodiments, the molecular weight of the charge shielding domain is at least 110%, at least 120%, at least 130%, at least 140%, at least 150%, at least 160%, at least 170% or at least 200% of molecular weight of the biologically active domain.

In some embodiments, the molecular weight of the charge shielding domain is about 25% to about 150% of the molecular weight of the biologically active domain. In some embodiments, the molecular weight of the charge shielding domain is about 50% to about 125% of the molecular weight of the biologically active domain. In some embodiments, the molecular weight of the charge-shielding domain is about 50% to about 100% of the molecular weight of the biologically active domain.

In some embodiments, the total molecular weight of the charge-shielded fusion protein is at least 50 kDa , at least 100 kDa, at least 120 kDa, or at least 150 kDa.

In some embodiments, the charge shielding domain adopts a random coil conformation. In some embodiments, the charge-shielding domain adopts a random coil conformation in an aqueous environment (e.g., an aqueous solution or an aqueous buffer). The presence of a random coil conformation can be determined using methods known in the art, in particular by means of spectroscopic techniques, such as circular dichroism (CD) spectroscopy. In some embodiments, the charge-shielding domain has a disordered structure. In some embodiments, the charge-shielding domain is unstructured.

In another embodiment, the charge-shielding domain is characterized in that is has greater than 90% random coil formation, or about 95%, or about 96%, or about 97%, or about 98%, or about 99% random coil formation as determined by GOR algorithm. In some embodiments, the charge-shielding domain has less than 20%, less than 15%, less than 10%, less than 5% or less than 3% alpha helices. In some embodiments, the charge-shielding domain has less than 20%, less than 15%, less than 10%, less than 5% or less than 3% beta sheets. In some embodiments, the charge-shielding domain has less than 2% alpha helices and less than 2% beta sheets as determined by the Chou-Fasman algorithm.

In another embodiment, the present invention provides fusion proteins, wherein the charge-shielding domain is characterized in that the sum of asparagine and glutamine residues is less than 10% of the total amino acid sequence of the charge-shielding domain, the sum of methionine and tryptophan residues is less than 2% of the total amino acid sequence of the charge-shielding domain, the charge-shielding domain sequence has less than 5% amino acid residues with a positive charge.

In another embodiment, the charge-shielding domain is characterized in that at least about 80%, or at least about 90%, or at least about 91%, or at least about 92%, or at least about 93%, or at least about 94%, or at least about 95%, or at least about 96%, or at least about 97%, or at least about 98%, or at least about 99% of the charge-shielding domain sequence consists of non-overlapping sequence motifs wherein each of the sequence motifs has about 9 to about 14 amino acid residues and wherein the sequence of any two contiguous amino acid residues does not occur more than twice in each of the sequence motifs consist of four to six types of amino acids selected from glycine (G), alanine (A), serine (S), threonine (T), glutamate (E) and proline (P).

In some embodiments, the charge-shielding domain increases the hydrodynamic radius of the fusion protein. The term “hydrodynamic radius” or “Stokes radius” is the effective radius (Rh in nm) of a molecule in a solution measured by assuming that it is a body moving through the solution and resisted by the solution's viscosity. In the embodiments of the invention, the hydrodynamic radius measurements of the fusion proteins correlate with the ‘apparent molecular weight factor’, which is a more intuitive measure. The “hydrodynamic radius” of a protein affects its rate of diffusion in aqueous solution as well as its ability to migrate in gels of macromolecules. The hydrodynamic radius of a protein is determined by its molecular weight as well as by its structure, including shape and compactness. Methods for determining the hydrodynamic radius are well known in the art, such as by the use of size exclusion chromatography (SEC), as described in U.S. Pat. Nos. 6,406,632 and 7,294,513. Most proteins have globular structure, which is the most compact three-dimensional structure a protein can have with the smallest hydrodynamic radius. Some proteins adopt a random and open, unstructured, or ‘linear’ conformation and as a result have a much larger hydrodynamic radius compared to typical globular proteins of similar molecular weight.

In some embodiments, the charge-shielding domain is able to enlarge the hydrodynamic radius of the fusion protein beyond the glomerular pore size of approximately 3-5 nm (corresponding to an apparent molecular weight of about 70 kDA) (Caliceti. 2003. Pharmacokinetic and biodistribution properties of poly(ethylene glycol)-protein conjugates. Adv Drug Deliv Rev 55:1261-1277), resulting in reduced renal clearance of circulating proteins. The hydrodynamic radius of a protein is determined by its molecular weight as well as by its structure, including shape or compactness. Methods for determining the hydrodynamic radius are well known in the art, such as by the use of size exclusion chromatography (SEC), as described in U.S. Pat. Nos. 6,406,632 and 7,294,513. Accordingly, in certain embodiments, the fusion protein has a hydrodynamic radius of at least about 5 nm, or at least about 8 nm, or at least about 10 nm, or 12 nm, or at least about 15 nm. In the foregoing embodiments, the large hydrodynamic radius conferred by the charge-shielding domain can lead to reduced renal clearance of the resulting fusion protein, leading to a corresponding increase in terminal half-life, an increase in mean residence time, and/or a decrease in renal clearance rate.

In some embodiments, the charge-shielding domain does not affect the function of the biologically active domain. In some embodiments, the biologically active domain retains at least 50%, at least 60% at least 70%, at least 80% at least 90% or at least 95% activity when fused to the charge-shielding domain.

In some embodiments, the charge-shielding domain increases the in vivo half-life of the fusion protein or a multimer (i.e. dimer, trimer, tetramer, hexamer, or octamer) of the charge-shielded fusion protein subunits. In some embodiments, the charge-shielding domain increases the in vivo half-life at least 10%, at least 20%, at least 30%, at least 40%, at least 50%, at least 60%, at least 70%, at least 80%, at least 90%, at least 100%, at least 150%, or at least 200% compared to the biologically active protein without the charge-shielding domain. In some embodiments, the charge-shielding domain increases the in vivo half-life at least 5 fold, at least 8 fold, at least 10 fold, at least 20 fold, or over 30 fold. In some embodiments, the charge-shielding domain increases the in vivo half-life 5 to 50 fold, 5 to 40 fold, 5 to 30 fold, or 5 to 20 fold.

In some embodiments, the charge-shielding domain is selected to confer an increase in the half-life for the fusion protein or a multimer of the fusion protein (i.e. dimer, trimer, tetramer, hexamer, or octamer) administered to an animal, compared to the corresponding biologically active domain not linked to the charge-shielding domain and administered at a comparable dose, of at least about two-fold longer, or at least about three-fold, or at least about four-fold, or at least about five-fold, or at least about six-fold, or at least about seven-fold, or at least about eight-fold, or at least about nine-fold, or at least about ten-fold, or at least about 15-fold, or at least a 20-fold, or at least a 40-fold, or at least a 80-fold, or at least a 100-fold or greater an increase in half-life compared to the biologically active domain not linked to the charge-shielding domain. In some embodiments, the invention provides a fusion protein that exhibits an increase of at least about 50%, or at least about 60%, or at least about 70%, or at least about 80%, or at least about 90%, or at least about a 100%, or at least about 150%, or at least about 200%, or at least about 300%, or at least about 500%, or at least about 1000%, or at least about a 2000% increase in AUC compared to the corresponding biologically active domain not linked to the charge-shielding domain and administered to an animal at a comparable dose. The pharmacokinetic parameters of a fusion protein can be determined by standard methods involving dosing, the taking of blood samples at times intervals, and the assaying of the protein using ELISA, HPLC, radioassay, or other methods known in the art or as described herein, followed by standard calculations of the data to derive the half-life and other PK parameters.

In addition, the fusion protein may have a half-life of at least about 5, 10, 12, 15, 24, 36, 48, 60, 72, 84 or 96 hours at a dose of about 25 μg protein/kg.

In some embodiments, the charge-shielding domain is a PAS domain. In some embodiments the PAS domain consists of proline, alanine, and/or serine residues. In some embodiments, the PAS domain comprises 10 to 1000 amino acids. In some embodiments, the PAS domain comprises 100 to 1000 amino acids. In some embodiments, the PAS domain comprises 200 to 800 amino acids. In some embodiments, the PAS domain comprises 200 to 700 amino acids. In some embodiments, the PAS domain comprises 200 to 600 amino acids. In some embodiments, the PAS domain comprises 200 to 400 amino acids.

In some embodiments, the charge-shielded fusion protein comprises a biologically active domain and a PAS domain. In some embodiments “PASylation” or “PASylated” as used herein means that a biologically active domain is fused to a PAS domain.

In some embodiments, the PAS domain comprises 10 to 100 or more proline and alanine amino acid residues, a total of 15 to 60 proline and alanine amino acid residues, a total of 15 to 45 proline and alanine amino acid residues, e.g. a total of 20 to about 40 proline and alanine amino acid residues, e.g. 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 proline and alanine amino acid residues. In a preferred aspect, said amino acid sequence consists of about 20 proline and alanine amino acid residues. In another preferred aspect, said amino acid sequence consists of about 40 proline and alanine amino acid residues.

The polypeptide consisting solely of proline and alanine amino acid residues may have a length of about 200 to about 400 proline and alanine amino acid residues. In other words the polypeptide may consist of about 200 to about 400 proline and alanine amino acid residues. In a preferred aspect, the polypeptide consists of a total of about 200 (e.g. 201) proline and alanine amino acid residues (i.e. has a length of about 200 (e.g. 201) proline and alanine amino acid residues) or the polypeptide consists of a total of about 400 (e.g. 401) proline and alanine amino acid residues (i.e. has a length of about 400 (e.g. 401) proline and alanine amino acid residues). In some embodiments, the charge-shielding domain consists of a random sequence of about 200 to about 400 proline and alanine residues.

The charge shielding domain may comprise a plurality of amino acid repeats, wherein said repeat consists of proline and alanine residues and wherein no more than 6 consecutive amino acid residues are identical. Particularly, the polypeptide may comprise or consist of the amino acid sequence AAPAAPAPAAPAAPAPAAPA (SEQ ID NO: 2) or circular permuted versions or (a) multimers(s) of the sequences as a whole or parts of the sequence.

(SEQ ID NO: 3)
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
A
(SEQ ID NO: 4)
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA

In some embodiments, the biologically active domain is a hormone. In some embodiments, the biologically active domain is an enzyme. In some embodiments, the biologically active domain is an immunoglobulin. In some embodiments, the biologically active domain is a therapeutic peptide. In some embodiments, the biologically active domain is a therapeutic polypeptide.

In some embodiments, the biologically active domain comprises one of the following or a variant, fragment or derivatives thereof: agouti related peptide, amylin, angiotensin, cecropin, bombesin, gastrin, including gastrin releasing peptide, lactoferin, antimicrobial peptides including but not limited to magainin, urodilatin, nuclear localization signal (NLS), collagen peptide, survivin, amyloid peptides, including f-amyloid, natiuretic peptides, peptide YY, neuroregenerative peptides and neuropeptides, including but not limited to neuropeptide Y, dynorphin, endomorphin, endothelin, enkaphalin, exendin, fibronectin, neuropeptide W and neuropeptide S, peptide T, melanocortin, amyloid precursor protein, sheet breaker peptide, CART 13 WO 2008/030968 PCT/US2007/077767 peptide, amyloid inhibitory peptide, prion inhibitory peptide, chlorotoxin, corticotropin releasing factor, oxytocin, vasopressin, cholecystokinin, secretin, thymosin, epidermal growth factor (EGF), vascular endothelial cell growth factor (VEGF), platelet-derived growth factor (PDGF), Insulin-like growth factor (IGF), fibroblast growth factors (aFGF, bFGF), pancreastatin, melanocyte stimulating hormone, osteocalcin, bradykinin, adrenomedullin, perinerin, metastatin, aprotinin, galanins, including galanin-like peptide, leptin, defensins, including but not limited to a-defensin and f defensin, salusin, and various venoms, including but not limited to conotoxin, decorsin, kurtoxin, anenomae venom, tarantula venom; natriuretic peptides including brain natriuretic peptide (B-type natriuretic peptide, or BNP), atrial natriuretic peptide, and vasonatrin; neurokinin A, neurokinin B; neuromedin; neurotensin; orexin, pancreatic polypeptide, pituitary adenylate cyclase activating peptide (PACAP), prolactin releasing peptide, proteolipid protein (PLP), somatostatin, TNF-a; Grehlin, Protein C (Xigris), SS1(dsFv)-PE38 and pseudomonas exotoxin protein, clotting factors, including antithrombin III and Coagulation Factor VIIA, Factor VIII, Factor IX, streptokinase, tissue plasminogen activators, urokinase, beta glucocerebrosidase and alpha-D-galactosidase, alpha L-iduronidase, alpha-1, 4-glucosidase, arylsulfatase B, iduronate-2-sulfatase, deoxyribunuclase I, human activated protein, follicle-stimulating hormone, chorionic gonadotropin, luteinizing hormone, somatropin, bone morphogenetic protein, nesiritide, parathyroid hormone, erythropoietin, keratinocyte growth factor, human granulocyte colony-stimulating factor (G-CSF), human granulocyte-macrophase colony stimulating factor (GM-CSF), alpha interferon, beta interferon, gamma interferon, interleukins, including IL-1, IL-iRa, IL-2, 11-4, IL-5, IL-6, IL-10, IL 11, IL-12, glycoprotein IIB/IIIA, immune globulins, including hepatitis B, gamma globulin, venoglobulin, hirudin, aprotinin, antithrombin III, alpha-i -proteinase inhibitor, filgrastim, and etanercept.

In another embodiment, the biologically active domain is an antibody or antigen, in connection with immunotherapy, or other therapeutic intervention.

In some embodiments, the biologically active domain comprises insulin A peptide, T20 peptide, interferon alpha 2B peptide, tobacco etch virus protease, small heterodimer partner orphan receptor, androgen receptor ligand binding domain, glucocorticoid receptor ligand binding domain, estrogen receptor ligand binding domain, G protein alpha Q, 1-deoxy-D-xylulose 5-phosphate reductoisomerase peptide, G protein alpha S, angiostatin (Ki-3), blue fluorescent protein (BFP), calmodulin (CalM), chloramphenicol acetyltransferase (CAT), green fluorescent protein (GFP), interleukin I receptor antagonist (IL-iRa), luciferase, tissue transglutaminase (tTg), morphine modulating neuropeptide 14 WO 2008/030968 PCT/US2007/077767 (MMN), neuropeptide Y (NPY), orexin-B, leptin, ACTH, calcitonin, adrenomedullin (AM), parathyroid hormone (PTH), defensin and growth hormone.

In some embodiments, the biologically active domain has a molecular weight that is less than 200 kDa. In some embodiments, the biologically active domain has a molecular weight that is less than about 150 kDa. In some embodiments, the biologically active domain has a molecular weight of less than about 100 kDa. In some embodiments, the biologically active domain has a molecular weight of less than about 70 kDa, which is the threshold value for kidney filtration. In some embodiments, the biologically active domain has a molecular weight of less than about 50 kDa.

In some embodiments, the biologically active domain has a molecular weight of about 20 to about 100 kDa. In some embodiments, the biologically active domain has a molecular weight of about 20 to about 70 kDa. In some embodiments, the biologically active domain has a molecular weight of about 30 to about 40 kDa.

In some embodiments, the biologically active domain can form a multimer. In some embodiments, the biologically active domain can form a dimer, trimer, tetramer, hexamer, or octamer. In some embodiments, the molecular weight of the multimeric biologically active domain is about 20 kDa to about 300 kDa, about 50 kDa to about 200 kDa, or about 100 kDa to about 200 kDa.

In some embodiments, the biologically active domain has a net charge in a neutral solution. In some embodiments, the biologically active domain has a pI that is not 7.0. In some embodiments, the biologically active domain has a pI of about 3.0 to about 6.0, about 4.0 to about 6.0, or about 5.0 to about 6.0. In some embodiments, the biologically active domain has a pI of about 8.0 to about 10.0, about 8.0 to about 9.0.

In some embodiments, the biologically active domain is an enzyme. In some embodiments, the biologically active domain is an asparaginase subunit. Recombinant type II asparaginase from Erwinia chrysanthemi, crisantaspase, is also known as Erwinase® and Erwinaze®. Recombinant asparaginase derived from E. coli is known by the names Colaspase®, Elspar®, Kidrolase®, Leunase®, and Spectrila®. Pegaspargase® is the name for a pegylated version of E. coli asparaginase. Crisantaspase is administered to patients with acute lymphoblastic leukemia, acute myeloid leukemia, and non-Hodgkin's lymphoma via intravenous, intramuscular, or subcutaneous injection.

In some embodiments, the asparaginase is an Erwinia chrysanthemi L-asparaginase type II (crisantaspase). In some embodiments, the asparaginase comprises the following amino acid sequence

(SEQ ID NO: 1)
ADKLPNIVILATGGTIAGSAATGTQTTGYKAGALGVDTLINAVPEVKKLA
NVKGEQFSNMASENMTGDVVLKLSQRVNELLARDDVDGVVITHGTDTVEE
SAYFLHLTVKSDKPVVFVAAMRPATAISADGPMNLLEAVRVAGDKQSRGR
GVMVVLNDRIGSARYITKTNASTLDTFKANEEGYLGVIIGNRIYYQNRID
KLHTTRSVFDVRGLTSLPKVDILYGYQDDPEYLYDAAIQHGVKGIVYAGM
GAGSVSVRGIAGMRKAMEKGVVVIRSTRTGNGIVPPDEELPGLVSDSLNP
AHARILLMLALTRTSDPKVIQEYFHTY.

In some embodiments, the asparagine is a recombinant E. coli asparaginase. E. coli produces two asparaginases, L-asparaginase type I and L-asparaginase type II. L-asparaginase type I, which has a low affinity for asparagine, is located in the cytoplasm. L-asparaginase type II is a tetrameric periplasmic enzyme with a high affinity for asparagine that is produced with a cleavable secretion leader sequence. U.S. Pat. Appl. No. US 2016/0060613, “Pegylated L-asparaginase” incorporated by reference in its entirety, describes common structural features of known L-asparaginases from bacterial sources. According to US 2016/0060613, all are homotetramers with four active sites between the N- and C-terminal domains of two adjacent monomers, all have a high degree of similarity in their tertiary and quaternary structures, and the sequences of the catalytic sites of L-asparaginases are highly conserved between Erwinia chrysanthemi, Erwinia carotovora, and E. coli L-asparaginase II.

In embodiments, the E. coli A-1-3 L-asparaginase type II comprises the amino acid sequence:

(SEQ ID NO: 5)
LPNITILATGGTIAGGGDSATKSNYTAGKVGVENLVNAVPQLKDIANVKG
EQVVNIGSQDMNDDVWLTLAKKINTDCDKTDGFVITHGTDTMEETAYFLD
LTVKCDKPVVMVGAMRPSTSMSADGPFNLYNAVVTAADKASANRGVLVVM
NDTVLDGRDVTKTNTTDVATFKSVNYGPLGYIHNGKIDYQRTPARKHTSD
TPFDVSKLNELPKVGIVYNYANASDLPAKALVDAGYDGIVSAGVGNGNLY
KTVFDTLATAAKNGTAVVRSSRVPTGATTQDAEVDDAKYGFVASGTLNPQ
KARVLLQLALTQTKDPQQIQQIFNQY

In some embodiments, the asparaginase is produced using the methods of the invention. This asparaginase is described, e.g., in U.S. Pat. No. 7,807,436, “Recombinant host for producing L-asparaginase II,” incorporated by reference herein in its entirety, wherein the sequence is set forth as SEQ ID NO: 5. The E. coli A-1-3 L-asparaginase type II also is described by Nakamura, N., et al., 1972, “On the Productivity and Properties of L-Asparaginase from Escherichia coli A-1-3,” Agricultural and Biological Chemistry, 36:12, 2251-2253, incorporated by reference herein. E. coli A-1-3 is derived from the E. coli HAP strain, which produces high levels of asparaginse, described in Roberts, J., et al., 1968, “New Procedures for Purification of L-Asparaginase with High Yield from Escherichia coli,” Journal of Bacteriology, 95:6, 2117-2123, incorporated by reference herein.

In embodiments, an L-asparaginase type II protein produced using the methods of the invention is the E. coli K-12 L-asparaginase type II enzyme, which has an amino acid sequence encoded by the ansB gene described by Jennings et al., 1990, J. Bacteriol. 172: 1491-1498 (GenBank No. M34277), both incorporated by reference herein (amino acid sequence set forth as

(SEQ ID NO: 6)
MEFFKKTALAALVMGFSGAALALPNITILATGGTIAGGGDSATKSNYTVG
KVGVENLVNAVPQLKDIANVKGEQVVNIGSQDMNDNVWLTLAKKINTDCD
KTDGFVITHGTDTMEETAYFLDLTVKCDKPVVMVGAMRPSTSMSADGPFN
LYNAVVTAADKASANRGVLVVMNDTVLDGRDVTKTNTTDVATFKSVNYGP
LGYIHNGKIDYQRTPARKHTSDTPPDVSKLNELPKVGIVYNYANASDLPA
KALVDAGYDGIVSAGVGNGNLYKSVFDTLATAAKTGTAVVRSSRVPTGAT
TQDAEVDDAKYGFVASGTLNPQKARVLLQLALTQTKDPQQIQQIFNQY
Or
(SEQ ID NO: 7)
LPNITILATGGTIAGGGDSATKSNYTVGKVGVENLVNAVPQLKDIANVKG
EQVVNIGSQDMNDNVWLTLAKKINTDCDKTDGFVITHGTDTMEETAYFLD
LTVKCDKPVVMVGAMRPSTSMSADGPFNLYNAVVTAADKASANRGVLVVM
NDTVLDGRDVTKTNTTDVATFKSVNYGPLGYIHNGKIDYQRTPARKHTSD
TPFDVSKLNELPKVGIVYNYANASDLPAKALVDAGYDGIVSAGVGNGNLY
KSVFDTLATAAKTGTAVVRSSRVPTGATTQDAEVDDAKYGFVASGTLNPQ
KARVLLQLALTQTKDPQQIQQIFNQY
(not including the leader sequence

U.S. Pat. No. 7,807,436 reports that, relative to the L-asparaginase type II enzyme from Merck & Co., Inc. (Elspar®) and L-asparaginase type II enzyme from Kyowa Hakko Kogyo Co., Ltd., the E. coli K12 enzyme subunit has Va127 in place of Ala27, Asn64 in place of Asp64, Ser252 in place of Thr252 and Thr263 in place of Asn263.

In embodiments, an L-asparaginase type II produced using the methods of the invention has an amino acid sequence set forth by Maita, T., et al, December 1974, “Amino acid sequence of L-asparaginase from Escherichia coli,” J. Biochem. 76(6):1351-4, incorporated by reference herein.

Recombinant type II asparaginase from E. coli is also known by the names Colaspase®, Elspar®, Kidrolase®, Leunase®, and Spectrila®. Pegaspargase® is the name for a pegylated version of E. coli asparaginase. Asparaginase is administered to patients with acute lymphoblastic leukemia, acute myeloid leukemia, and non-Hodgkin's lymphoma via intravenous, intramuscular, or subcutaneous injection.

In some embodiments, the fusion protein comprises an asparaginase subunit with at least 80%, at least 90%, at least 95%, at least 96%, at least 97%, at least 98% or at least 99% amino acid identity with SEQ ID NO:7. In some embodiments, the fusion protein comprises an asparaginase subunit comprising SEQ ID NO:7 with one, two, three, four, five, six, seven, eight, nine, or ten amino acid substitutions. In some embodiments, the amino acid substitutions are conservative substitutions. In some embodiments, the fusion protein comprises an asparaginase subunit comprising SEQ ID NO:7 with one, two, three, four, five, six, seven, eight, nine, or ten amino acid insertions or deletions.

Substitutions include conservative amino acid substitutions. A “conservative amino acid substitution” is one in which the amino acid residue is replaced with an amino acid residue having a similar side chain, or physicochemical characteristics (e.g., electrostatic, hydrogen bonding, isosteric, hydrophobic features). The amino acids may be naturally occurring or unnatural Families of amino acid residues having similar side chains are known in the art. These families include amino acids with basic side chains (e.g. lysine, arginine, histidine), acidic side chains (e.g., aspartic acid, glutamic acid), uncharged polar side chains (e.g., glycine, asparagine, glutamine, serine, threonine, tyrosine, methionine, cysteine), nonpolar side chains (e.g., alanine, valine, leucine, isoleucine, proline, phenylalanine, tryptophan), β-branched side chains (e.g., threonine, valine, isoleucine) and aromatic side chains (e.g., tyrosine, phenylalanine, tryptophan, histidine). Substitutions may also include non-conservative changes.

Erwinia chrysanthemi NCPPB 1066 (Genbank Accession No. CAA32884, described by, e.g., Minton, et al., 1986, “Nucleotide sequence of the Erwinia chrysanthemi NCPPB 1066 L-asparaginase gene,” Gene 46(1), 25-35, each incorporated herein by reference in its entirety), either with or without signal peptides and/or leader sequences.

In some embodiments, the fusion protein comprises an asparaginase from Dickeya chrysanthemi. In some embodiments, the asparaginase comprises the amino acid sequence

(SEQ ID NO: 8)
ADKLPNIVILATGGTIAGSAATGTQTTGYKAGALGVDTLINAVPEVKKLA
NVKGEQFSNMASENMTGDVVLKLSQRVNELLARDDVDGVVITHGTDTVEE
SAYFLHLTVKSDKPVVFVAAMRPATAISADGPMNLLEAVRVAGDKQSRGR
GVMVVLNDRIGSARYITKTNASTLDTFKANEEGYLGVIIGNRIYYQNRID
KLHTTRSVFDVRGLTSLPKVDILYGYQDDPEYLYDAAIQHGVKGIVYAGM
GAGSVSVRGIAGMRKAMEKGVVVIRSTRTGNGIVPPDEELPGLVSDSLNP
AHARILLMLALTRTSDPKVIQEYFHTY

In some embodiments, the fusion protein comprises the amino acids sequence

(SEQ ID NO: 9)
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
AADKLPNIVILATGGTIAGSAATGTQTTGYKAGALGVDTLINAVPEVKKL
ANVKGEQFSNMASENMTGDVVLKLSQRVNELLARDDVDGVVITHGTDTVE
ESAYFLHLTVKSDKPVVFVAAMRPATAISADGPMNLLEAVRVAGDKQSRG
RGVMVVLNDRIGSARYITKTNASTLDTFKANEEGYLGVIIGNRIYYQNRI
DKLHTTRSVFDVRGLTSLPKVDILYGYQDDPEYLYDAAIQHGVKGIVYAG
MGAGSVSVRGIAGMRKAMEKGVVVIRSTRTGNGIVPPDEELPGLVSDSLN
PAHARILLMLALTRTSDPKVIQEYFHTY

In some embodiments, the fusion protein comprises the amino acid sequence

(SEQ ID NO: 10)
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
AAPAAPAPAAPAAPAPAAPAAAPAAPAPAAPAAPAPAAPAAAPAAPAPAA
PAAPAPAAPAAAPAAPAPAAPAAPAPAAPAAAPAAPAPAAPAAPAPAAPA
AADKLPNIVILATGGTIAGSAATGTQTTGYKAGALGVDTLINAVPEVKKL
ANVKGEQFSNMASENMTGDVVLKLSQRVNELLARDDVDGVVITHGTDTVE
ESAYFLHLTVKSDKPVVFVAAMRPATAISADGPMNLLEAVRVAGDKQSRG
RGVMVVLNDRIGSARYITKTNASTLDTFKANEEGYLGVIIGNRIYYQNRI
DKLHTTRSVFDVRGLTSLPKVDILYGYQDDPEYLYDAAIQHGVKGIVYAG
MGAGSVSVRGIAGMRKAMEKGVVVIRSTRTGNGIVPPDEELPGLVSDSLN
PAHARILLMLALTRTSDPKVIQEYFHTY

### IV. Production of Charge-Shielded Recombinant Erwinia Fusion Proteins

In some embodiments, the method comprises expressing and purifying a charge-shielded asparaginase fusion protein. In some embodiments, the method comprises expressing a type II asparaginase fusion protein. In some embodiments, the asparaginase is a is an Erwinia chrysanthemi L-asparaginase type II (crisantaspase). In some embodiments, the asparaginase fusion protein is expressed an a prokaryotic host cell. In some embodiments, the asparaginase fusion protein is expressed in a Pseudomonas fluorescens host cell. In some embodiments, the Pseudomonadales host cell is deficient in the expression of one or more native asparaginases. In some embodiments, the deficiently expressed native asparaginase is a type I asparaginase. In some embodiments, the deficiently expressed native asparaginase is a type II asparaginase. In some embodiments, the Pseudomonadales host cell is deficient in the expression of one or more proteases. In some embodiments, the Pseudomonadales host cell overexpresses one or more folding modulators. In some embodiments, the Pseudomonadales host cell is deficient in the expression of one or more native asparaginases, is deficient in the expression of one or more proteases and/or overexpresses one or more folding modulators. U.S. Pat. No. 10,787,671 provides methods for producing recombinant Erwinia asparaginase.

In its native host, Erwinia chrysanthemi, crisantaspase is produced in the periplasm. The present invention provides methods that allow production of high levels of soluble and/or active crisantaspase in the cytoplasm of the host cell. In embodiments, methods provided herein yield high levels of soluble and/or active crisantaspase in the cytoplasm of a Pseudomonadales, Pseudomonad, Pseudomonas, or Pseudomonas fluorescens host cell.

In some embodiments, the charge-shielded fusion protein is purified from a periplasmic releasate. In some embodiments, nucleic acid encoding the charge-shielded fusion protein comprise a periplasm secretion leader sequence.

In some embodiments, osmotic shock is used to produce a periplasmic releasate. In some embodiments, cells are incubated with lysozyme to produce a periplasmic releasate. In some embodiments, cells are sonicated to produce a periplasmic releasate. In some embodiments, cells are incubated with lysozyme and sonicated to produce a periplasmic releasate.

In some embodiments, to release the charge-shielded fusion protein from the periplasm, chemicals such as chloroform (Ames et al. (1984) J. Bacteriol., 160: 1181-1183), guanidine-HCl, and Triton X-100 (Naglak and Wang (1990) Processes including Enzyme Microb. Technol., 12: 603-611) have been used. However, these chemicals are not inert and can adversely affect many recombinant protein products or subsequent purification procedures. Glycine treatment of E. coli cells, resulting in increased permeability of the outer membrane, has also been reported to release periplasmic contents (Ariga et al. (1989) J. Ferm. Bioeng., 68: 243-246) . The most widely used method of recombinant protein periplasmic release is osmotic shock (Nosal and Heppel (1966) J. Biol. Chem., 241: 3055-3062; Neu and Heppel (1965) J. Biol. Chem., 24 0: 3685-3692), hen eggwhite (HEW) lysozyme/ethylenediaminetetraacetic acid (EDTA) treatment (Neu and Heppel (1964) J. Biol. Chem., 239: 3893-3900; Witholt e t al. (1976) Biochim Biophys. Acta, 443: 534-544; Pierce et al. (1995) ICheme Research. Event, 2: 995-997), and HEW lysozyme/osmotic shock combined treatment (French et al. (1996) Enzyme and Microb. Tech., 19: 332-338). The French method involves resuspension of the cells in fractionation buffer followed by recovery of the periplasmic fraction, and an osmotic shock is performed immediately after lysozyme treatment.

Typically, these procedures involve initial disruption in media that stabilizes osmotic pressure, followed by selective release in non-stabilized media. The composition of these media (pH, protective agent) and the disruption method used (chloroform, HEW lysozyme, EDTA, sonication) depend on the specific procedure reported. HEW using zwitterionic surfactant instead of EDTA A variation on lysozyme/EDTA treatment is described in Statel et al. (1994) Veterinary Microbiol., 38: 307-314. For a general review of the use of intracellular lytic enzyme systems to destroy E. coli, see Dabora and Cooney (1990) in Advances in Biochemical Engineering/Biotechnology, Vol. 43, A. Fiechter, ed. (Springer-Verlag: Berlin), pp. See 11-30.

In some embodiments, the charge-shielded asparaginase fusion protein is expressed in an expression construct, such as a plasmid, without a secretion signal. Inducible promoter sequences are used to regulate expression of crisantaspase in accordance with the methods herein. In embodiments, inducible promoters useful in the methods herein include those of the family derived from the lac promoter (i.e. the lacZ promoter), especially the tac and trc promoters described in U.S. Pat. No. 4,551,433 to DeBoer, as well as Ptac16, Ptac17, PtacII, PlacUV5, and the T7lac promoter. In one embodiment, the promoter is not derived from the host cell organism. In certain embodiments, the promoter is derived from an E. coli organism. In some embodiments, a lac promoter is used to regulate expression of crisantaspase from a plasmid. In the case of the lac promoter derivatives or family members, e.g., the tac promoter, an inducer is IPTG (isopropyl-β-D-1-thiogalactopyranoside, also called “isopropylthiogalactoside”). In certain embodiments, IPTG is added to culture to induce expression of crisantaspase from a lac promoter in a Pseudomonas host cell.

An expression construct useful in practicing the methods herein include, in addition to the protein coding sequence, the following regulatory elements operably linked thereto: a promoter, a ribosome binding site (RBS), a transcription terminator, and translational start and stop signals.

Pseudomonas and closely related bacteria are generally part of the group defined as “Gram(−) Proteobacteria Subgroup 1” or “Gram-Negative Aerobic Rods and Cocci” (Bergey's Manual of Systematics of Archaea and Bacteria (online publication, 2015)). Pseudomonas host strains are described in the literature, e.g., in U.S. Pat. App. Pub. No. 2006/0040352, cited above.

“Gram-negative Proteobacteria Subgroup 1” also includes Proteobacteria that would be classified in this heading according to the criteria used in the classification. The heading also includes groups that were previously classified in this section but are no longer, such as the genera Acidovorax, Brevundimonas, Burkholderia, Hydrogenophaga, Oceanimonas, Ralstonia, and Stenotrophomonas, the genus Sphingomonas (and the genus Blastomonas, derived therefrom), which was created by regrouping organisms belonging to (and previously called species of) the genus Xanthomonas, the genus Acidomonas, which was created by regrouping organisms belonging to the genus Acetobacter as defined in Bergey's Manual of Systematics of Archaea and Bacteria (online publication, 2015). In addition hosts include cells from the genus Pseudomonas, Pseudomonas enalia (ATCC 14393), Pseudomonas nigrifaciensi (ATCC 19375), and Pseudomonas putrefaciens (ATCC 8071), which have been reclassified respectively as Alteromonas haloplanktis, Alteromonas nigrifaciens, and Alteromonas putrefaciens. Similarly, e.g., Pseudomonas acidovorans (ATCC 15668) and Pseudomonas testosteroni (ATCC 11996) have since been reclassified as Comamonas acidovorans and Comamonas testosteroni, respectively; and Pseudomonas nigrifaciens (ATCC 19375) and Pseudomonas piscicida (ATCC 15057) have been reclassified respectively as Pseudoalteromonas nigrifaciens and Pseudoalteromonas piscicida. “Gram-negative Proteobacteria Subgroup 1” also includes Proteobacteria classified as belonging to any of the families: Pseudomonadaceae, Azotobacteraceae (now often called by the synonym, the “Azotobacter group” of Pseudomonadaceae), Rhizobiaceae, and Methylomonadaceae (now often called by the synonym, “Methylococcaceae”). Consequently, in addition to those genera otherwise described herein, further Proteobacterial genera falling within “Gram-negative Proteobacteria Subgroup 1” include: 1) Azotobacter group bacteria of the genus Azorhizophilus; 2) Pseudomonadaceae family bacteria of the genera Cellvibrio, Oligella, and Teredinibacter; 3) Rhizobiaceae family bacteria of the genera Chelatobacter, Ensifer, Liberibacter (also called “Candidatus liberibacter”), and Sinorhizobium; and 4) Methylococcaceae family bacteria of the genera Methylobacter, Methylocaldum, Methylomicrobium, Methylosarcina, and Methylosphaera.

The host cell, in some cases, is selected from “Gram-negative Proteobacteria Subgroup 16.” “Gram-negative Proteobacteria Subgroup 16” is defined as the group of Proteobacteria of the following Pseudomonas species (with the ATCC or other deposit numbers of exemplary strain(s) shown in parenthesis): Pseudomonas abietaniphila (ATCC 700689); Pseudomonas aeruginosa (ATCC 10145); Pseudomonas alcaligenes (ATCC 14909); Pseudomonas anguilliseptica (ATCC 33660); Pseudomonas citronellolis (ATCC 13674); Pseudomonas flavescens (ATCC 51555); Pseudomonas mendocina (ATCC 25411); Pseudomonas nitroreducens (ATCC 33634); Pseudomonas oleovorans (ATCC 8062); Pseudomonas pseudoakaligenes (ATCC 17440); Pseudomonas resinovorans (ATCC 14235); Pseudomonas straminea (ATCC 33636); Pseudomonas agarici (ATCC 25941); Pseudomonas alcaliphila; Pseudomonas alginovora; Pseudomonas andersonii; Pseudomonas asplenii (ATCC 23835); Pseudomonas azelaica (ATCC 27162); Pseudomonas beyerinckii (ATCC 19372); Pseudomonas borealis; Pseudomonas boreopolis (ATCC 33662); Pseudomonas brassicacearum; Pseudomonas butanovora (ATCC 43655); Pseudomonas cellulosa (ATCC 55703); Pseudomonas aurantiaca (ATCC 33663); Pseudomonas chlororaphis (ATCC 9446, ATCC 13985, ATCC 17418, ATCC 17461); Pseudomonas fragi (ATCC 4973); Pseudomonas lundensis (ATCC 49968); Pseudomonas taetrolens (ATCC 4683); Pseudomonas cissicola (ATCC 33616); Pseudomonas coronafaciens; Pseudomonas diterpeniphila; Pseudomonas elongata (ATCC 10144); Pseudomonas flectens (ATCC 12775); Pseudomonas azotoformans; Pseudomonas brenneri; Pseudomonas cedrella; Pseudomonas corrugata (ATCC 29736); Pseudomonas extremorientalis; Pseudomonas fluorescens (ATCC 35858); Pseudomonas gessardii; Pseudomonas libanensis; Pseudomonas mandelii (ATCC 700871); Pseudomonas marginalis (ATCC 10844); Pseudomonas migulae; Pseudomonas mucidolens (ATCC 4685); Pseudomonas orientalis; Pseudomonas rhodesiae; Pseudomonas synxantha (ATCC 9890); Pseudomonas tolaasii (ATCC 33618); Pseudomonas veronii (ATCC 700474); Pseudomonas frederiksbergensis; Pseudomonas geniculata (ATCC 19374); Pseudomonas gingeri; Pseudomonas graminis; Pseudomonas grimontii; Pseudomonas halodenitrificans; Pseudomonas halophila; Pseudomonas hibiscicola (ATCC 19867); Pseudomonas huttiensis (ATCC 14670); Pseudomonas hydrogenovora; Pseudomonas jessenii (ATCC 700870); Pseudomonas kilonensis; Pseudomonas lanceolata (ATCC 14669); Pseudomonas lini; Pseudomonas marginata (ATCC 25417); Pseudomonas mephitica (ATCC 33665); Pseudomonas denitrificans (ATCC 19244); Pseudomonas pertucinogena (ATCC 190); Pseudomonas pictorum (ATCC 23328); Pseudomonas psychrophila; Pseudomonas filva (ATCC 31418); Pseudomonas monteilii (ATCC 700476); Pseudomonas mosselii; Pseudomonas oryzihabitans (ATCC 43272); Pseudomonas plecoglossicida (ATCC 700383); Pseudomonas putida (ATCC 12633); Pseudomonas reactans; Pseudomonas spinosa (ATCC 14606); Pseudomonas balearica; Pseudomonas luteola (ATCC 43273); Pseudomonas stutzeri (ATCC 17588); Pseudomonas amygdali (ATCC 33614); Pseudomonas avellanae (ATCC 700331); Pseudomonas caricapapayae (ATCC 33615); Pseudomonas cichorii (ATCC 10857); Pseudomonas ficuserectae (ATCC 35104); Pseudomonas fuscovaginae; Pseudomonas meliae (ATCC 33050); Pseudomonas syringae (ATCC 19310); Pseudomonas viridiflava (ATCC 13223); Pseudomonas thermocarboxydovorans (ATCC 35961); Pseudomonas thermotolerans; Pseudomonas thivervalensis; Pseudomonas vancouverensis (ATCC 700688); Pseudomonas wisconsinensis; and Pseudomonas xiamenensis. In one embodiment, the host cell for expression of crisantaspase is Pseudomonas fluorescens.

The host cell, in some cases, is selected from “Gram-negative Proteobacteria Subgroup 17.” “Gram-negative Proteobacteria Subgroup 17” is defined as the group of Proteobacteria known in the art as the “fluorescent Pseudomonads” including those belonging, e.g., to the following Pseudomonas species: Pseudomonas azotoformans; Pseudomonas brenneri; Pseudomonas cedrella; Pseudomonas cedrina; Pseudomonas corrugata; Pseudomonas extremorientalis; Pseudomonas fluorescens; Pseudomonas gessardii; Pseudomonas libanensis; Pseudomonas mandelii; Pseudomonas marginalis; Pseudomonas migulae; Pseudomonas mucidolens; Pseudomonas orientalis; Pseudomonas rhodesiae; Pseudomonas synxantha; Pseudomonas tolaasii; and Pseudomonas veronii.

In embodiments a host strain useful for expressing a charge-shielded crisantaspase fusion protein, in the methods of the invention is a Pseudomonas host strain, e.g., P. fluorescens, having a protease deficiency or inactivation (resulting from, e.g., a deletion, partial deletion, or knockout) and/or overexpressing a folding modulator, e.g., from a plasmid or the bacterial chromosome. In embodiments, the host strain expresses the auxotrophic markers pyrF and proC, and has a protease deficiency and/or overexpresses a folding modulator. In embodiments, the host strain expresses any other suitable selection marker known in the art. In any of the above embodiments, an asparaginase, e.g., a native Type I and/or Type II asparaginase, is inactivated in the host strain. In one embodiment, the methods herein comprise expression of recombinant charge-shielded crisantaspase fusion protein from a construct that has been optimized for codon usage in a strain of interest. In embodiments, the strain is a Pseudomonas host cell, e.g., Pseudomonas fluorescens. Methods for optimizing codons to improve expression in bacterial hosts are known in the art and described in the literature.

Growth conditions useful in the methods herein often comprise a temperature of about 4° C. to about 42° C. and a pH of about 5.7 to about 8.8. When an expression construct with a lacZ promoter or derivative thereof is used, expression is often induced by adding IPTG to a culture at a final concentration of about 0.01 mM to about 1.0 mM. II. Charge-Shielded Proteins

As described elsewhere herein, inducible promoters are often used in the expression construct to control expression of the recombinant charge-shielded crisantaspase fusion protein, e.g., a lac promoter. In the case of the lac promoter derivatives or family members, e.g., the tac promoter, the effector compound is an inducer, such as a gratuitous inducer like IPTG (isopropyl-β-D-1-thiogalactopyranoside, also called “isopropylthiogalactoside”). In embodiments, a lac promoter derivative is used, and charge-shielded crisantaspase fusion protein expression is induced by the addition of IPTG to a final concentration of about 0.01 mM to about 1.0 mM, when the cell density has reached a level identified by an OD575 of about 25 to about 160.

After adding an inducing agent, cultures are often grown for a period of time, for example about 24 hours, during which time the recombinant charge-shielded crisantaspase fusion protein is expressed. After adding an inducing agent, a culture is often grown for about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, about 24 hr, about 36 hr, or about 48 hr. After an inducing agent is added to a culture, the culture is grown for about 1 to 48 hrs, about 1 to 24 hrs, about 10 to 24 hrs, about 15 to 24 hrs, or about 20 to 24 hrs. Cell cultures are often concentrated by centrifugation, and the culture pellet resuspended in a buffer or solution appropriate for the subsequent lysis procedure.

In embodiments, cells are disrupted using equipment for high pressure mechanical cell disruption (which are available commercially, e.g., Microfluidics Microfluidizer, Constant Cell Disruptor, Niro-Soavi homogenizer or APV-Gaulin homogenizer). Cells expressing charge-shielded crisantaspase fusion proteins are often disrupted, for example, using sonication. Any appropriate method known in the art for lysing cells are often used to release the soluble fraction. For example, in embodiments, chemical and/or enzymatic cell lysis reagents, such as cell-wall lytic enzyme and EDTA, are often used. Use of frozen or previously stored cultures is also contemplated in the methods herein. Cultures are sometimes OD-normalized prior to lysis. For example, cells are often normalized to an OD600 of about 10, about 11, about 12, about 13, about 14, about 15, about 16, about 17, about 18, about 19, or about 20.

Centrifugation is performed using any appropriate equipment and method. Centrifugation of cell culture or lysate or periplasmic releasate for the purposes of separating a soluble fraction from an insoluble fraction is well-known in the art. For example, lysed cells are sometimes centrifuged at 20,800×g for 20 minutes (at 4° C.), and the supernatants removed using manual or automated liquid handling. The pellet (insoluble) fraction is resuspended in a buffered solution, e.g., phosphate buffered saline (PBS), pH 7.4. Resuspension is often carried out using, e.g., equipment such as impellers connected to an overhead mixer, magnetic stir-bars, rocking shakers, etc.

In one embodiment, fermentation is used in the methods of producing recombinant charge-shielded crisantaspase fusion protein . The expression system according to the present disclosure is cultured in any fermentation format. For example, batch, fed-batch, semi-continuous, and continuous fermentation modes may be employed herein. In embodiments, the fermentation medium may be selected from among rich media, minimal media, and mineral salts media. In other embodiments either a minimal medium or a mineral salts medium is selected. In certain embodiments, a mineral salts medium is selected.

Fermentation may be performed at any scale. The expression systems according to the present disclosure are useful for recombinant protein expression at any scale. Thus, e.g., microliter-scale, milliliter scale, centiliter scale, and deciliter scale fermentation volumes may be used, and 1 Liter scale and larger fermentation volumes are often used.

In embodiments, the methods herein are used to obtain a yield of soluble recombinant charge-shielded crisantaspase fusion protein, e.g., monomer or tetramer, of about 1% to about 70% total cell protein. In certain embodiments, the yield of soluble recombinant charge-shielded crisantaspase fusion protein is about 1% total cell protein, about 2% total cell protein, about 3% total cell protein, about 4% total cell protein, about 5% total cell protein, about 8% total cell protein, about 10% total cell protein, about 15% total cell protein, about 20% total cell protein, about 25% total cell protein, about 30% total cell protein, about 35% total cell protein, about 40% total cell protein, about 41% total cell protein, about 42% total cell protein, about 43% total cell protein, about 44% total cell protein, about 45% total cell protein, about 46% total cell protein, about 47% total cell protein, about 48% total cell protein, about 49% total cell protein, about 50% total cell protein, about 51% total cell protein, about 52% total cell protein, about 53% total cell protein, about 54% total cell protein, about 55% total cell protein, about 56% total cell protein, about 57% total cell protein, about 58% total cell protein, about 59% total cell protein, about 60% total cell protein, about 65% total cell protein, about 70% total cell protein, about 75% total cell protein, about 80% total cell protein, about 85% total cell protein, or about 90% total cell protein.

In some embodiments, the yield of soluble recombinant charge-shielded crisantaspase fusion protein is about 1% to about 70% total cell protein, about 1% to about 50% total cell protein, about 1% to about 20% total cell protein, about 1% to about 10% total cell protein, about 1% to about 5% total cell protein, about 1% to about 3% total cell protein, about 20% to about 55% total cell protein, about 20% to about 60% total cell protein, about 20% to about 65% total cell protein, about 20% to about 70% total cell protein, about 20% to about 75% total cell protein, about 20% to about 80% total cell protein, about 20% to about 85% total cell protein, about 20% to about 90% total cell protein, about 25% to about 90% total cell protein, about 30% to about 90% total cell protein, about 35% to about 90% total cell protein, about 40% to about 90% total cell protein, about 45% to about 90% total cell protein, about 50% to about 90% total cell protein, about 55% to about 90% total cell protein, about 60% to about 90% total cell protein, about 65% to about 90% total cell protein, about 70% to about 90% total cell protein, about 75% to about 90% total cell protein, about 80% to about 90% total cell protein, about 85% to about 90% total cell protein, about 1% to about 5% total cell protein, about 2% to about 5% total cell protein, about 5% to about 10% total cell protein, about 20% to about 35% total cell protein, about 20% to about 30% total cell protein, or about 20% to about 25% total cell protein. In some embodiments, the yield of soluble recombinant charge-shielded crisantaspase fusion protein is about 20% to about 40% total cell protein.

In embodiments, the methods herein are used to obtain a yield of soluble recombinant charge-shielded crisantaspase fusion protein, e.g., monomer or tetramer, of about 1 gram per liter to about 50 grams per liter. In certain embodiments, the yield of soluble recombinant charge-shielded crisantaspase fusion protein is about 0.25, about 0.5 gram per liter, about 1 gram per liter, about 2 grams per liter, about 3 grams per liter, about 4 grams per liter, about 5 grams per liter, about 6 grams per liter, about 7 grams per liter, about 8 grams per liter, about 9 grams per liter, about 10 gram per liter, about 11 grams per liter, about 12 grams per liter, about 13 grams per liter, about 14 grams per liter, about 15 grams per liter, about 16 grams per liter, about 17 grams per liter, about 18 grams per liter, about 19 grams per liter, about 20 grams per liter, about 21 grams per liter, about 22 grams per liter, about 23 grams per liter about 24 grams per liter, about 25 grams per liter, about 26 grams per liter, about 27 grams per liter, about 28 grams per liter, about 30 grams per liter, about 35 grams per liter, about 40 grams per liter, about 45 grams per liter about 50 grams per liter.

In some embodiments, the yield of soluble recombinant charge-shielded crisantaspase fusion protein is about 0.1 to about 6 grams per liter, about 0.25 to about 4 grams per liter, about 0.5 to about 2 grams per liter, about 1 gram per liter to about 5 grams per liter, about 0.75 gram to about 10 grams per liter, about 0.75 gram per liter to about 3 grams per liter, about 0.75 grams per liter to about 2 grams per liter, about 0.75 grams per liter to about 1.5 grams per liter, about 0.5 grams per liter to about 15 grams per liter, about 0.5 grams per liter to about 10 grams per liter, about 0.5 grams per liter to about 8 grams per liter, about 0.5 grams per liter to about 6 grams per liter, about 0.5 grams per liter to about 6 grams per liter, about 0.1 grams per liter to about 20 grams per liter, about 0.1 grams per liter to about 10 grams per liter, about 0.1 grams per liter to about 8 grams per liter, about 0.1 grams per liter to about 5 grams per liter, about 0.1 grams per liter to about 3 grams per liter, about 0.1 grams per liter to about 25 grams per liter liter to about 25 grams per liter, or about 24 grams per liter to about 25 grams per liter.

In embodiments, the yield ratio of cytoplasmically produced soluble recombinant crisantaspase to periplasmically produced soluble recombinant charge-shielded crisantaspase fusion protein obtained under similar or substantially similar conditions is about 1 to about 5. In embodiments, the yield ratio of cytoplasmically produced soluble recombinant charge-shielded crisantaspase fusion protein to periplasmically produced soluble recombinant charge-shielded crisantaspase fusion protein obtained under similar or substantially similar conditions is at least about 1.

### V. Production of Charge-Shielded Recombinant E. Coli Asparagine Fusion Proteins

It would be understood by one of skill in the art that a production host strain useful in the methods of the present invention can be generated using a publicly available host cell, for example, P. fluorescens MB101, e.g., by inactivating the pyrF gene, and/or the Type I L-asparaginase gene, and/or the Type II L-asparaginase gene, using any of many appropriate methods known in the art and described in the literature. It is also understood that a prototrophy restoring plasmid can be transformed into the strain, e.g., a plasmid carrying the pyrF gene from strain MB214 using any of many appropriate methods known in the art and described in the literature. Additionally, in such strains, proteases can be inactivated and folding modulator overexpression constructs introduced, using methods well known in the art.

In embodiments a host strain useful for expressing an asparaginase, e.g., an E. coli asparaginase type II, in the methods of the invention is a Pseudomonas host strain, e.g., P. fluorescens, having a protease deficiency or inactivation (resulting from, e.g., a deletion, partial deletion, or knockout) and/or overexpressing a folding modulator, e.g., from a plasmid or the bacterial chromosome. In any embodiments, the host strain expresses the auxotrophic markers pyrF and proC, and has a protease deficiency and/or overexpresses a folding modulator. In embodiments, the host strain expresses any other suitable selection marker known in the art. In any of the above embodiments, an asparaginase, e.g., a native Type I and/or Type II asparaginase, is inactivated in the host strain.

As described elsewhere herein, inducible promoters are often used in the expression construct to control expression of the recombinant asparaginase, e.g., a lac promoter. In the case of the lac promoter derivatives or family members, e.g., the tac promoter, the effector compound is an inducer, such as a gratuitous inducer like IPTG (isopropyl-β-D-1-thiogalactopyranoside, also called “isopropylthiogalactoside”). In embodiments, a lac promoter derivative is used, and asparaginase expression is induced by the addition of IPTG to a final concentration of about 0.01 mM to about 1.0 mM, when the cell density has reached a level identified by an OD575 of about 25 to about 160.

In embodiments, cells are disrupted using equipment for high pressure mechanical cell disruption (which are available commercially, e.g., Microfluidics Microfluidizer, Constant Cell Disruptor, Niro-Soavi homogenizer or APV-Gaulin homogenizer). Cells expressing asparaginase are often disrupted, for example, using sonication. Any appropriate method known in the art for lysing cells are often used to release the soluble fraction. For example, in embodiments, chemical and/or enzymatic cell lysis reagents, such as cell-wall lytic enzyme and EDTA, are often used. Use of frozen or previously stored cultures is also contemplated in the methods herein. Cultures are sometimes OD-normalized prior to lysis. For example, cells are often normalized to an OD600 of about 10, about 11, about 12, about 13, about 14, about 15, about 16, about 17, about 18, about 19, or about 20.

### VI. Compositions Comprising Charge-Shielded Proteins

Also provided herein are compositions comprising charge-shielded proteins. In some embodiments, the composition is a pharmaceutical composition. In some embodiments, the pharmaceutical composition comprises the charge-shielded protein and one or more pharmaceutically acceptable carriers.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. Suitable carriers may comprise any material which, when combined with the biologically active protein of the invention, retains the biological activity of the biologically active protein (see Remington's Pharmaceutical Sciences (1980) 16th edition, Osol, A. Ed). Preparations for parenteral administration may include sterile aqueous or non-aqueous solutions, suspensions, and emulsions). The buffers, solvents and/or excipients as employed in context of the pharmaceutical composition are preferably “physiological” as defined herein above. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles may include sodium chloride solution, Ringer's dextrose, dextrose and sodium chloride, lactated Ringer's, or fixed oils. Intravenous vehicles may include fluid and nutrient replenishes, electrolyte replenishers (such as those based on Ringer's dextrose), and the like. Preservatives and other additives may also be present including, for example, antimicrobials, anti-oxidants, chelating agents, and inert gases and the like. In addition, the pharmaceutical composition of the present invention might comprise proteinaceous carriers, like, e.g., serum albumin or immunoglobulin, preferably of human origin.

In some embodiments, provided herein is a composition comprising the charge-shielded protein purified protein following a first hydrophobic interaction chromatography column has purity of up to, greater than, or about 80%, about 85%, about 90%, or about 95%.

In some embodiments, provided herein is a composition comprising the charge-shielded protein at a purity of at least 90%, at least 91%, at least 92%, at least 93%, at least 94%, at least 95%, at least 96%, at least 97%, at least 98% or at least 99%.

In some embodiments, the composition comprises the charge-shielded protein at a concentration of at least 1 mg/mL. In some embodiments, the composition comprises the charge-shielded protein at a concentration of at least 5 mg/mL, at least 10 mg/mL, at least 20 mg/mL, at least 50 mg/mL, at least 100 mg/mL or at least 300 mg/mL. In some embodiments, the composition comprises the charge-shielded protein at a concentration of 1 to 50 mg/mL.

### VI. Methods of Treatment

In some embodiments, provided herein are methods of treating an individual comprising administering a composition comprising a charge-shielded protein to an individual in need thereof. In some embodiments, the individual has cancer or a neoplastic disease. In some embodiments, the individual has leukemia, lymphoma, or myeloma. In some embodiments, the individual has acute lymphoblastic lymphoma. In some embodiments, the disease is a metabolic disease. In some embodiments, the disease is hormone deficiency-related disorders, auto-immune disease, cancer, anemia, neovascular diseases, infectious/inflammatory diseases, thrombosis, myocardial infarction or diabetes.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Expression and Purification of PASylated Asparaginase Using Ion Exchange Chromatography as the Capture Step

This example demonstrates the expression of a charge-shielded PASylated asparaginase fusion protein (e.g., PF745) from periplasmic releasate.

A recombinant crisantaspase (RC) (asparaginase from Erwinia chrysanthemi), genetically fused at its amino terminus to a 200-amino acid polypeptide sequence comprised entirely of proline and alanine residues (PA200), was successfully expressed. The PA200 fusion partner was designed by XL-Protein GmbH using their proprietary technology, PASylation®, which extends the half-life of biopharmaceuticals by applying an intrinsically disordered protein as a biological alternative to PEGylation. The PA200-RC fusion protein was expressed in Pseudomonas fluorescens to generate recombinant PA200-RC fusion protein, meeting specified purity and potency targets.

The fusion protein was named PF745, and construction was initiated by cloning of the PA200-RC DNA fusion in P. fluorescens. Initial screening of 1,040 expression strains at a 96-well scale demonstrated that successful expression of a soluble PA200-RC protein monomer. The strain STR58751 (expressing PA200-RC protein localized in the periplasm) was chosen for production of PF745, based on high titer expression of soluble monomer under multiple fermentation induction conditions, reproducibly low N-term truncation profile (<2%), and results from identity, activity, and purity methods. PF745 was expressed and released from cells by osmotic extraction, due to the selection of a periplasmic expression strain during strain engineering. The osmotic extraction was optimized to maximize product release from cells, while minimizing host-cell contaminant (e.g., host cell protein (HCP)) release.

Efforts were made to perform capture of PF745 by ion exchange chromatography (IEX) from a periplasmic releasate as a first chromatography capture step.

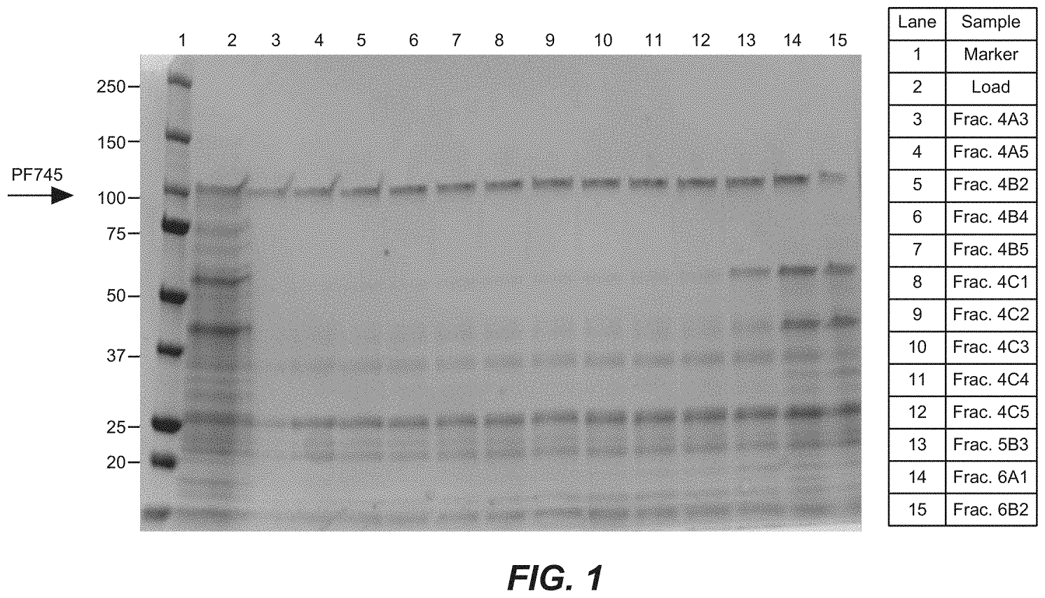

Anion exchange chromatography (AEX) was performed following osmotic shock extraction of PF745 from STR58751. The extract was adjusted to pH 9 and a conductivity of 0.8 mS/cm, and loaded onto a POROS 50 HQ AEX column, running in flow through mode with a load ratio of 0.82 g paste/mL resin. However, breakthrough of HCP was observed in fraction 6A1 (Lane 14 of FIG. 1), indicating that AEX does not provide sufficient enrichment of the target protein (e.g., a charge-shielded fusion protein, PF745) when performed as an initial purification step.

Attempts to capture PF745 from a cytoplasmic expression strain using CEX as a primary capture/purification step were also unsuccessful, and this approach was therefore not attempted using osmotic shock from STR58751. Binding to CEX as a second column was also not acceptable, indicating that its use as a capture step, in the presence of even higher levels of HCP contaminants than a second column would encounter, would also be unsuccessful in enriching the target.

Both AEX and CEX capture steps showed either no capture or extremely low binding capacity. This suggests that shielding from PA200 moieties effectively masked charge on PF745.

### Example 2. Purification of PF745 Using Size Exclusion Chromatography Followed by CEX

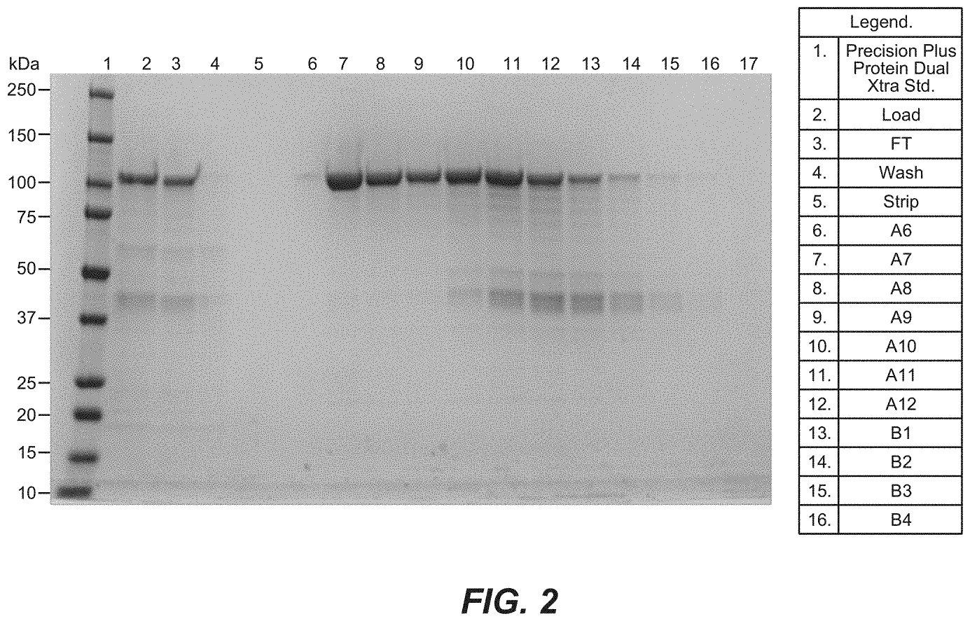

Periplasmic extract from STR58751 was adjusted to 2 M ammonium sulfate, and loaded onto a SephacryIS500 resin for size exclusion chromatography (SEC). The flow through fractions containing the target molecule were pooled and concentrated using a 100 kDa concentrator device, the concentrated pool was adjusted to 0.5 mS/cm resin, and was loaded on a POROS XS cation change resin in a bind and elute mode. Binding of the target was observed (see fractions A6-B4, lanes 6-16 of FIG. 2), however binding capacity was low and most of the target was observed in the flow through fraction (Lane 3, FT of FIG. 2). The volume of the load and flow through fractions were almost identical, and equal volumes (16 μL) were loaded on an SDS-PAGE gel. The purity of the target protein in the load was 53.5%, as determined by densitometry, and the purity of the target protein in the flow through was 52.9%, indicating that most of the PF745 protein did not bind to the CEX column and stayed in the flow through.

These results indicated that a higher load purity is required for CEX capture of charge-shielded fusion proteins (e.g., PF745).

### Example 3. Expression and Purification of a Charge-Shielded Fusion Protein

This example demonstrates the successful purification of a charge-shielded fusion protein (e.g., PF745) from a periplasmic releasate. In particular, this example demonstrates the sequential use of hydrophobic interaction chromatography (HIC), anion exchange chromatography (AEX), and cation exchange chromatography (CEX), to increase the purity of PF745 from a periplasmic releasate.

Hydrophobic Interaction Chromatography

Six HIC resins were tested. Toyopearl Butyl-650M demonstrated high binding capacity and acceptable purity, and was therefore used as an exemplary HIC column. Osmotic extracts were adjusted to 2.5 M NaCl with a final conductivity of 178±15 mS/cm, and pH 6.0±0.2. Adjusted periplasmic releasate was filtered and immediately loaded to the Toyopearl Butyl-650M capture column, at a load ratio of <0.17 g paste/ mL resin.

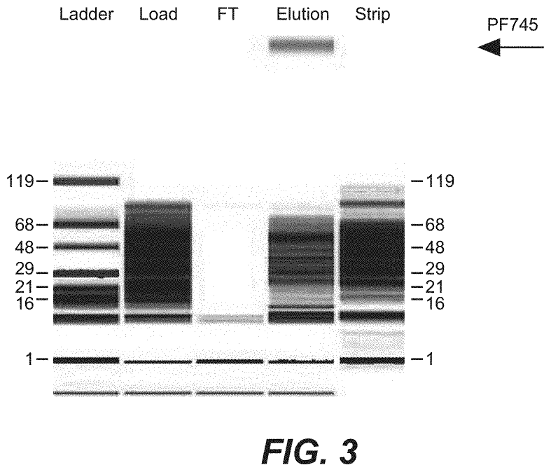

In order to obtain sufficient protein, this capture column was cycled 8-10 times for each run for a total of 26 times. This column consistently yielded 8-9 mg PF745 per gram paste loaded (as measured by A279), with purity values of approximately 75% and 60% as measured by RP-HPLC and SE-HPLC, respectively. An SDS-CGE image from a representative HIC step using a Butyl-650M resin is shown in FIG. 3, to demonstrate the purification afforded by this capture step.

### Anion Exchange Chromatography

Recovered concentrate from ultrafiltration/diafiltration (UF/DF) 1 following HIC was further purified using an AEX chromatography step, to determine whether purity could be increased. A POROS HQ resin was used as an exemplary AEX resin. To reduce the risk of potential deamidation, UF/DF 1-recovered concentrate was adjusted to a pH of 9.0±0.2 immediately before loading on the POROS HQ column. Similarly, collected flow through and wash pools were also adjusted to a pH of 6.0±0.2, immediately upon completion of the POROS HQ chromatography step.

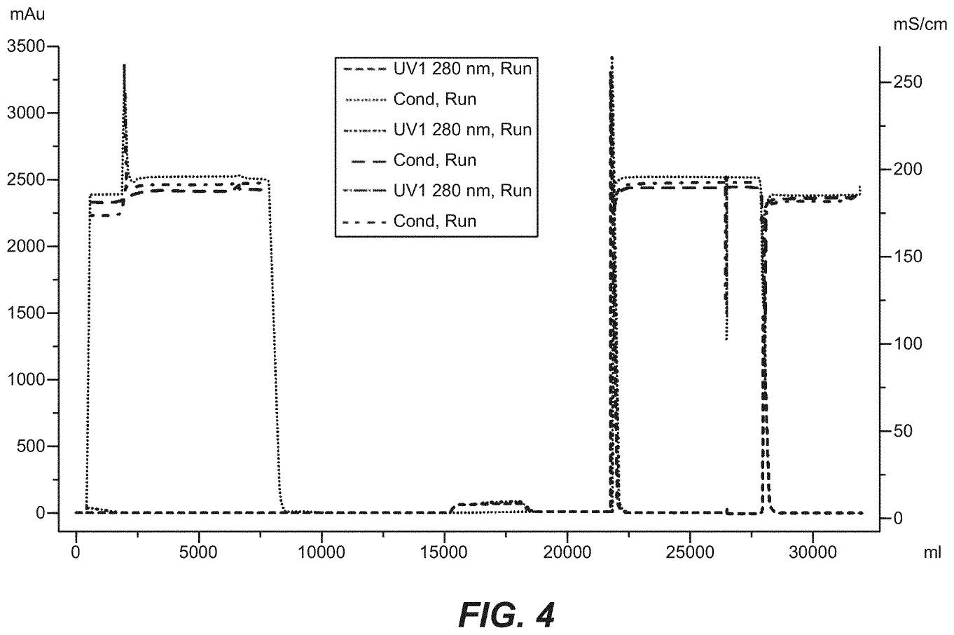

Each lot was cycled between 4 and 6 time to process all material without exceeding a loading ratio of 5 mg PF745/mL resin. Despite cycling of the column, chromatograms indicated that AEX chromatography was consistent and reproducible. Furthermore, all runs resulted in a consistent AEX step yield of 22-25% (FIG. 4). While this yield appears low, this is more indicative of impurities being removed, rather than product being lost. This is readily seen by comparing areas of the strip peak to the flow through+wash product peak. Additionally, further evidence of impurity removal is seen in the RP-HPLC and SE-HPLC values, increasing to 90% and 80% respectively, after this AEX step. Finally, the HCP content for all AEX eluates decreased to less than 120 ppm, corroborating the HPLC results of an increasingly pure sample.

Thus, the AEX step consistently performed well and removed a significant amount of impurities following the HIC step.

Cation Exchange Chromatography

Recovered concentrate from a UF/DF 2 step was purified using a CEX chromatography step, to test whether purity could be further increased. POROS XS was used as an exemplary CEX resin. Each lot required between 3 and 6 cycles of the CEX step to process all material without exceeding a loading ratio of 5 mg PF745/mL resin. Despite cycling the column, chromatograms indicated that CEX chromatography was consistent and reproducible.

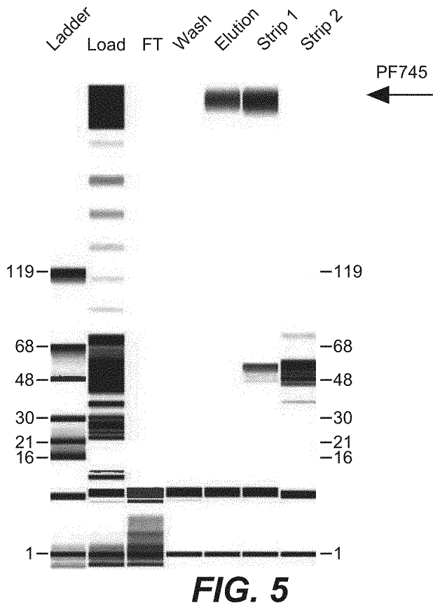

Furthermore, all runs resulted in a consistent CEX step recovery of 48-54%, with product of purity greater than 94% and 100% by RP-HPLC and SE-HPLC, respectively. These purity values were further supported by CGE analysis, which shows that the PF745 was effectively separated from the impurities remaining in the load material, resulting in a highly pure eluate (FIG. 5). Finally, the HCP content of all CEX eluates was less than 3 ppm, corroborating the HPLC results of a highly pure sample.

Thus, the CEX step consistently performed well and removed a significant amount of impurities, producing material that exceeded target purity values, to increase purity to an even greater extent following HIC and AEX chromatography steps.

Conclusion

Sequential steps of HIC, AEX chromatography, and CEX chromatography, in that order, can reliably be used for the efficient purification of charge-shielded proteins, such as PF745, from a periplasmic releasate.

### Example 4. Hydrophobic Interaction Chromatography for PASylated Asparaginase Purification

This example demonstrates a method of hydrophobic interaction chromatography (HIC) for the purification of a charge-shielded fusion protein from a periplasmic releasate. In particular, this example demonstrates HIC resin screening and the use of HIC for enrichment of target charge-shielded fusion proteins.

PF745 (a PASylated asparaginase) was expressed and released from cells by osmotic extraction, as described, due to the selection of a periplasmic expression strain during strain engineering. Upon releasing and clarifying material from cells, capture was tested using hydrophobic interaction chromatography (HIC).

Resins

A plate-based resin screening was performed to demonstrate that thirteen hydrophobic interaction resins (Table 1) have recovery of bind/elute or flow-through purification using 96-well filter plates (Agilent, Cat# 200957-100) and the Biosero Automation System, which includes a Tecan Freedom Evo 200 liquid handling system and a Bionex HiG4 automated centrifuge.

TABLE 1
Hydrophobic interaction chromatography resins that
may be used for the first chromatography step

| Resin | Manufacturer | Catalog # |
|---|---|---|
| POROS Benzyl Ultra | Thermo Scientific | A32569 |
| POROS Benzyl | Thermo Scientific | A32563 |
| Hexyl-650C | Tosoh Bioscience | 0019026 |
| Capto Phenyl (high sub) | GE Healthcare | 17-5451-02 |
| Butyl-650M | Tosoh Bioscience | 0019802 |
| Phenyl-600M | Tosoh Bioscience | 0021888 |
| Capto Phenyl ImpRes | GE Healthcare | 17-5484-03 |
| Phenyl Sepharose HP | GE Healthcare | 17-1082-01 |
| Octyl Sepharose 4 FF | GE Healthcare | 17-0946-02 |
| Capto Octyl | GE Healthcare | 17-5465-02 |
| PPG-600M | Tosoh Bioscience | 0021301 |
| POROS Ethyl | Thermo Scientific | A32557 |

To prepare the resin plates, 50 μL of a 50% slurry of each resin was pipetted to each well of a 96-well plate with the Tecan for a target 25 μL resin per well. A high-hydrophobicity resin plate was prepared with one resin per row in the following order from highest to lowest hydrophobicity: Benzyl Ultra, Benzyl, Hexyl-650C, Capto Phenyl, Butyl-650M, Phenyl-600M, Capto Phenyl ImpRes, and Phenyl Sepharose HP. A low-hydrophobicity resin plate was prepared with one resin per row in the following order from highest to lowest hydrophobicity: Octyl Sepharose 4 PP, Capto Octyl, PPG-600M, Ethyl, and Butyl-650M.

The plates were centrifuged to allow the slurry liquid to filter through. Table 2 includes the chromatography steps that started with stripping the resin with water. The plates were centrifuged after each cycle of pipetting. The resins were then equilibrated with respective equilibration buffers. PF745 intermediate from osmotic shock and ultrafiltration/diafiltration (UF/DF) 1 was adjusted with kosmotrope spike solutions to kosmotrope concentrations corresponding with the equilibration buffers. Then, the adjusted PF745 intermediates were diluted with the corresponding equilibration buffers to an equivalent of 167 mg paste per mL solution, and filtered through Sartobran P 0.45/0.2 μm filters. After loading 150 μL (targeting 1 g paste per mL resin), the filtrate was collected for flow-through assessment. The wash and elution filtrates were similarly collected in separate plates. The flow-through and elution were analyzed via SDS-CGE.

TABLE 2
Chromatography steps for resin screening

| | | | | | Volume | | Pi- |
|---|---|---|---|---|---|---|---|
| | | | | | per | | petting |
| | Solution by columns. . . | | | | well | # of | up and |
| Phase | 1-3 | 4-6 | 7-9 | 10-12 | (μL) | cycles | down |
| Strip | Milli-Q water | | | | 150 | 3 | 10 |
| | | | | | | | times |
| EQ | 0.25M | 0.5M | 2M | 3M | 150 | 2 | 10 |
| | Na₂SO₄, | Na₂SO₄, | Na₂SO₄, | Na₂SO₄, | | | times |
| | 20 mM | 19 mM | 24 mM | 246 mM | | | |
| | NaP, | NaP, | NaP, | NaP, | | 1 | 30 |
| | pH 6.2 | pH 6.2 | pH 6.2 | pH 6.2 | | | min |

| | | | | |
|---|---|---|---|---|
| Load | UF/DF 1 intermediate adjusted | 150 | 1 | 120 |
| | to match EQ buffer | | | min |
| Wash | Same as EQ | 150 | 1 | 10 |
| | | | | times |
| Elution | Milli-Q water | 150 | 1 | 10 |
| | | | | times |

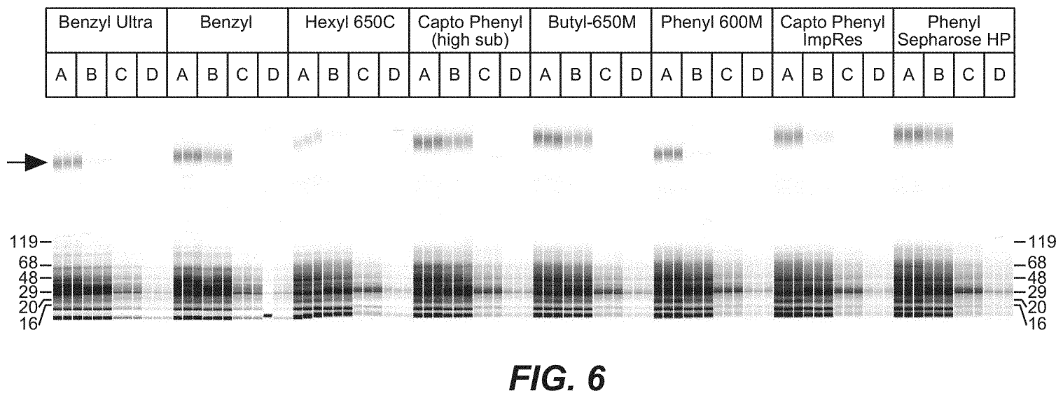

FIG. 6 shows that four resins (Benzyl Ultra, Hexyl-650C, Phenyl-600M, and Capto Phenyl ImpRes) bound the target under most conditions, as evidenced by very little detectable PF745 band in the flow-through. All resins demonstrated poor binding with 0.25 M sodium sulfate. In cases where PF745 did not bind, the flow-through does not demonstrate significantly improved purity relative to the load.

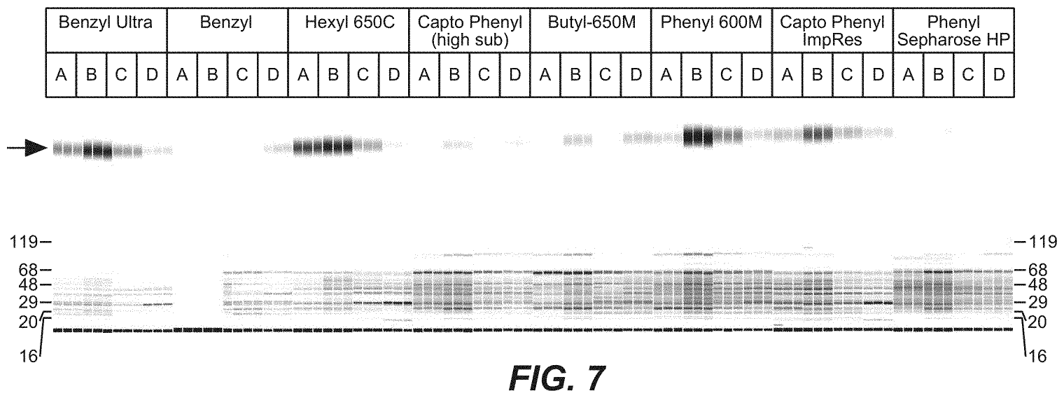

The resins identified as having the highest binding based on flow-through also demonstrated the best elution recoveries—Benzyl Ultra, Hexyl-650C, Phenyl-600M, and Capto Phenyl ImpRes (FIG. 7). The binding condition generating the best recoveries was 0.5 M ammonium sulfate (triplicate “B” columns in the figure). In addition to promising recoveries, the elutions showed a significant increase in SDS-CGE purity, as evidenced by a decrease in low molecular weight (LMW) bands.

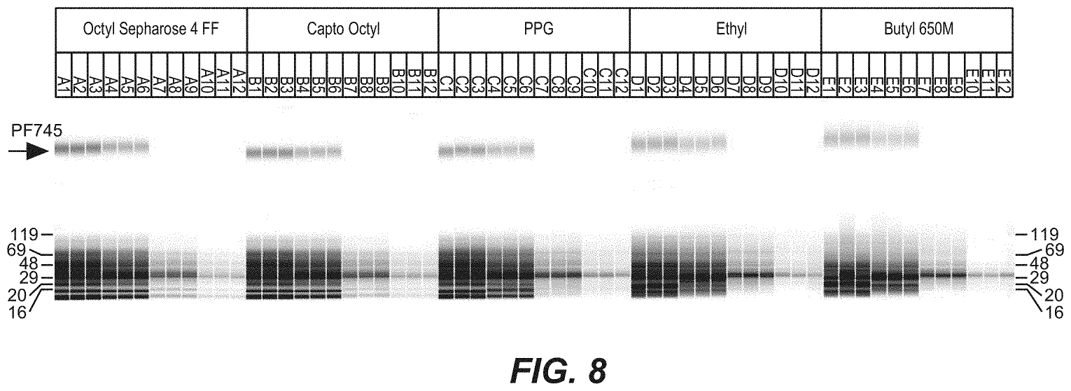

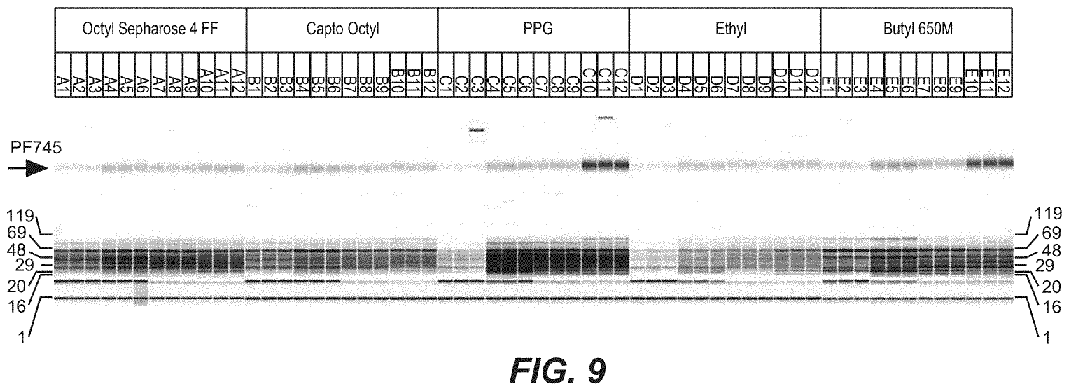

In contrast, the low-hydrophobicity resins bound small amounts of PF745 in both 0.25 M sodium sulfate and 0.5 M ammonium sulfate, as evidenced by PF745 bands in the flow-throughs of those conditions (FIG. 8); additionally, there was no separation of PF745 from impurities. Most conditions yielded low recovery (FIG. 9). Only 3 M NaCl load combined with PPG or Butyl-650M resins yielded significant visible bands.

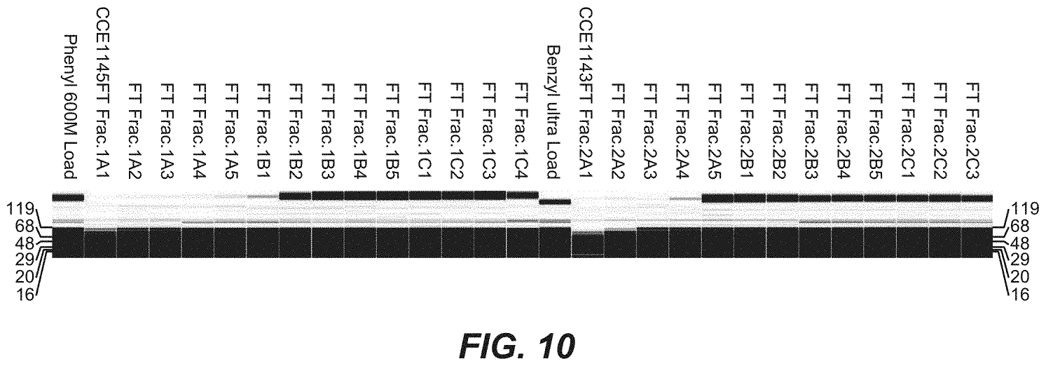

Benzyl Ultra, Hexyl-650C, Phenyl-600M, and Capto Phenyl ImpRes were tested at 6.1-7.5 mL column scale with the same load material and load challenge of ˜6 g paste per mL resin. An SDS-CGE image from the run on Phenyl-600M demonstrated PF745 enrichment early in the elution gradient (Fractions 1A6-1B5, FIG. 10) and separation from impurities (primarily between 16 to 68 kDa) that eluted later in the gradient; the load purity measured about 1% compared to 50-75% in elution fractions (FIG. 10). The load flow-through was not collected, but the low and high flow wash fractions indicate little breakthrough near the end of loading, suggesting that most of the PF745 loaded was bound.

Table 3 illustrates the elution yield and purity. Phenyl-600M displayed the most efficient capture properties.

TABLE 3
Elution yield and purity of HIC capture
resins that may be used for the first
chromatography step

| | | Elution | | | |
|---|---|---|---|---|---|
| | | pool | By RP-HPLC. . . | | |
| | | volume | Concentration | Yield | Purity |
| CCE# | Resin | (mL) | (mg/mL) | (mg) | (%) |
| 1144 | Phenyl-600M | 36 | 0.69 | 24.8 | 96 |
| 1142 | Benzyl Ultra | 48 | 0.49 | 23.5 | 98 |
| 1146 | Hexyl-650C | 48 | 0.22 | 10.6 | 84 |
| 1148 | Capto Phenyl ImpRes | 12 | 0.02 | 0.2 | 9 |

In the initial Phenyl-600M and Benzyl Ultra dynamic binding capacity (DBC) trial, a significant breakthrough was observed earlier in the Benzyl Ultra flow-through than that of Phenyl-600M. Phenyl-600M demonstrated binding equivalent to about 9.7 g paste per mL resin. Phenyl-600M showed a 30% higher binding capacity.

The conditions that were effective for HIC purification of PF745, using an exemplary Phenyl-600M HIC resin, included a 0.60 M ammonium sulfate load maximized resin capacity. Such elution ammonium sulfate concentration was maintained at 0.40 M ammonium sulfate (60-75 mS/cm) to provide high target recovery. Load and elution pH was set at 5.9±0.1, and consistently captured PF745 material, and provided a SDS-CGE purity of 40-60% after a single HIC capture step.

Ultimately, an exemplary HIC capture method, using an exemplary Phenyl-600M resin, was created based on several factors (e.g., 1) pH of EQ, Load, Wash, and Elution; 2) ammonium sulfate concentration of EQ, Load, Wash, and Elution; 3) Load challenge; and, 4) ammonium sulfate concentration of Elution). The exemplary HIC method is briefly described in Table 4, and defined by phase, buffer/solution, column volume (CV), and low flow rate (cm/h).

TABLE 4
Exemplary HIC method using Phenyl-600M

| | | | | LFR |
|---|---|---|---|---|
| Phase | Buffer/Solution | CV | | (cm/h) |
| Pre-EQ | Milli-Q water | 3 | | 150 |
| Equilibration | 20 mM sodium phosphate, | 4 | | 150 |
| | 2 mM EDTA, 0.60M | | | |
| | ammonium sulfate, pH 5.9 | | | |
| Load | Depth-filtered UF/DF 1 | Challenge: | | 75 |
| | intermediate adjusted | ≤7 | g paste/mL | |
| | to 0.60M ammonium | | | |
| | sulfate, pH 5.9 | | | |
| Wash | 20 mM sodium phosphate, | 1 | | 75 |
| | 2 mM EDTA, 0.60M | 4 | | 150 |
| | ammonium sulfate, pH 5.9 | | | |
| Elution | 20 mM sodium phosphate, | 4 | | 150 |
| | 2 mM EDTA, 0.40M | | | |
| | ammonium sulfate, pH 5.9 | | | |
| Strip | Milli-Q water | 6 | | 150 |
| Sanitization | 1N NaOH | 4 | (upflow) | 150 |
| | | | | (+60 min hold) |
| Rinse | Milli-Q water | 3 | (upflow) | 150 |
| Cleaning | 5M Urea | 5 | (upflow) | 150 |
| Rinse | Milli-Q water | 4 | (upflow) | 150 |
| Storage | 0.1N NaOH | 4 | (upflow) | 150 |

### Conclusion

This example shows that HIC chromatography can be used as a capture step to efficiently purify charge-shielded proteins, such as PF745, from a periplasmic releasate achieving over 90% purity after a single chromatography step (e.g., capture step).

### Example 5. Anion Exchange Chromatography for PASylated Asparaginase Purification

This example demonstrates a method of anion exchange chromatography (AEX) for the purification of a charge-shielded fusion protein from a cell lysate.

Anion exchange chromatography was tested as a second chromatography step following the first HIC chromatography step. Five AEX resins (Table 5) were packed in 0.66 cm Omnifit columns for initial screening (PCE1245-1249).

TABLE 5
Anion exchange chromatography resins that may
be used for the second chromatography step

| Resin | Manufacturer | Catalog # |
|---|---|---|
| GigaCap Q 650M | Tosoh Bioscience | 0021855 |
| Super Q-650M | Tosoh Bioscience | 0043205 |
| NH2-750F | Tosoh Bioscience | 0023438 |
| POROS 50 HQ | Thermo Scientific | 82078 |
| POROS XQ | Thermo Scientific | 82074 |

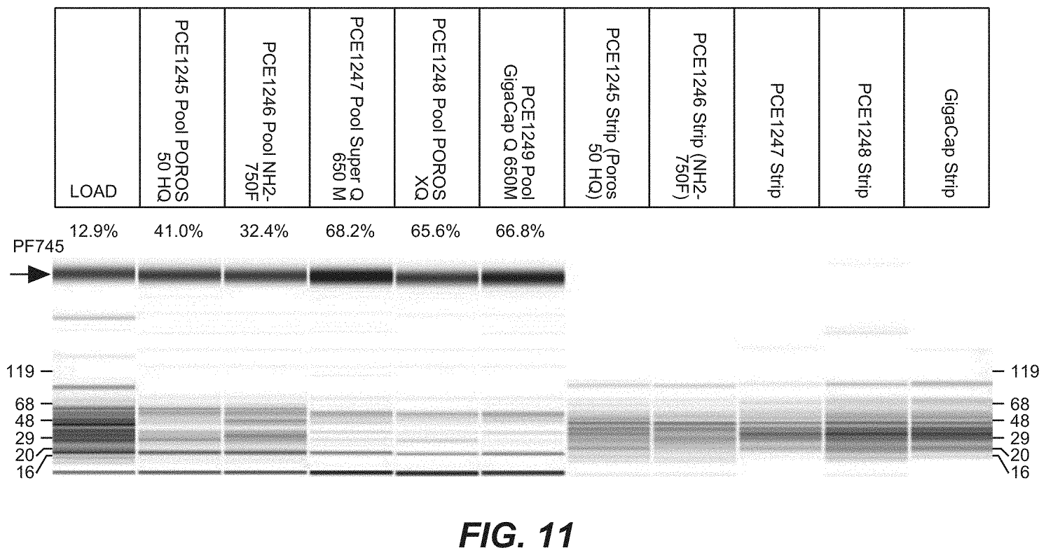

FIG. 11 shows that the GigaCap Q-650M, POROS XQ, and Super Q-650M flow-through pools had significantly higher SDS-CGE purity (65.6-68.2%) than those of POROS 50 HQ and NH2-750F (32.4-41.0%). The strips from all five runs did not contain any measurable PF745, indicating high recovery of target and no unintended binding for all tested AEX resins. The POROS HQ and NH2-750F flow-through pools had significantly higher HCP levels than those of the other runs, which aligns with the lower SDS-CGE purities (FIG. 11). The Super Q-650M flow-through pool had >5X the HCP content of the pools from GigaCap Q-650M and POROS XQ. GigaCap Q-650M and POROS XQ produced the highest purity with the lowest HCP.

Based on the SDS-CGE integration of flow-through fractions (FIG. 11), the GigaCap Q-650M flow-through achieved higher purity than POROS XQ and POROS 50 HQ. The purity of the latter two resins declined through loading while purity remained relatively consistent across loading for GigaCap Q-650M. The flow-through pools achieved similar purity results as shown in Table 6. Therefore, all of these resins are suitable for use in the second CEX chromatography step.

TABLE 6
Flow-through product quality from resins that
may be used for the second chromatography step

| | | % purity by. . . | | HCP |
|---|---|---|---|---|
| PCE# | Resin | RP-HPLC | SE-HPLC | (ng/mg) |
| 1251 | POROS 50 HQ | 88.6 | 93.4 | N/A |
| 1252 | POROS XQ | 89.5 | 93.1 | 1625 |
| 1253 | GigaCap Q-650M | 89.1 | 93.3 | 1099 |

No significant differences were observed in RP-HPLC and SE-HPLC purity between the runs. GigaCap Q-650M demonstrated the ability to maintain SDS-CGE purity at the given challenge and the lowest measured HCP level.

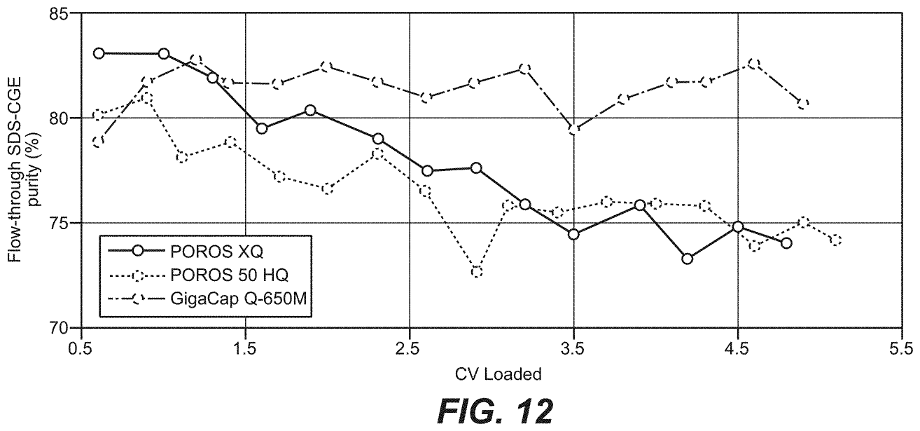

GigaCap Q-650M and POROS XQ demonstrated similar performance with respect to SDS-CGE purity of flow-through fractions (FIG. 12). Both resins achieved and maintained high purity relative to the load at <60% up to loading of 57 mg/mL. The rapidly declining purity between 14-16 CV is due to lower PF745 concentrations, as the load transitioned to the wash. The GigaCap Q-650M resin maintained a slightly SDS-CGE higher purity throughout loading, and additionally resulted in lower HCP levels in the previous experiment.

A screening experiment assessing five AEX resins demonstrated that GigaCap Q-650M achieved high purification performance. Conditions effective for protein purification by AEX chromatography included a load pH 9.0 and 1.0 mS/cm, and was robust to load concentrations between 2 and 6 mg/mL. Additional experiments demonstrated that 1.0 mS/cm load conductivity resulted in high yield, without diminishing load stability. Conditions suitable for AEX chromatography included a load pH of 9.0±0.1, load conductivity of 1.0±0.1 mS/cm, load concentration of 2-6 mg/mL, load held at pH 9 for <6 h load challenge between 6-25 mg/mL, and flow-through titrated to pH 6.0±0.1 within 6 h.

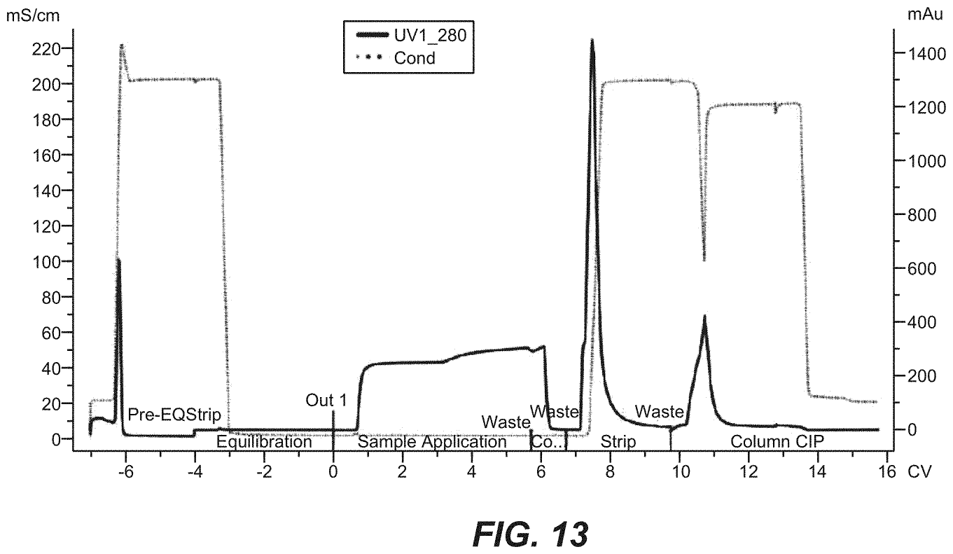

Using these conditions for purification generated consistent recovery, with flow-through purity of ≥85% by RP-HPLC, ≥80% by SEC-HPLC, HCP level of <1000 ng/mg, and HCDNA levels of <500 pg/mg. A representative chromatograph of the exemplary AEX chromatography step is illustrated in FIG. 13.

TABLE 7
Exemplary AEX method using GigaCap Q-650M

| | | | | LFR |
|---|---|---|---|---|
| Phase | Buffer/Solution | CV | | (cm/h) |
| Pre-EQ | 50 mM Tris, 3M | 3 | | 150 |
| | NaCl, pH 8.0 | | | |
| Equilibration | 20 mM Tris, 5.0 mM | 4 | | 150 |
| | NaCl, 2 mM EDTA, | | | |
| | pH 9.0, 1.0 mS/cm | | | |
| Load | UF/DF 2 intermediate | Challenge: | | 75 |
| | adjusted to pH 9.0 ± 0.1, | 6-25 | mg/mL | |
| | 1.0 ± 0.1 mS/cm, | | | |
| | 2-6 mg/mL | | | |
| Wash | Same as EQ | 1 | | 75 |
| Strip | 50 mM Tris, 3M NaCl, | 3 | | 150 |
| | pH 8.0 | | | |
| Sanitization | 1N NaOH | 3 | (upflow) | 150 |
| | | | | (+60-minute |
| | | | | hold) |
| Storage | 0.1N NaOH | 3 | (upflow) | 150 |

To minimize possible deamidation, it is recommended that the load adjustment to pH 9.0 occur immediately prior to loading for AEX, and that flow-through adjustment to pH 6.0 occur as soon as possible after collection.

### Conclusion

An AEX chromatography step, following an initial HIC step, improved the purity of the charge-shielded protein PF745.

### Example 6. Cation Exchange Chromatography for PASylated Asparaginase Purification

This example demonstrates a method of cation exchange chromatography (CEX) as a third chromatography step for the purification of a charge-shielded fusion protein (e.g., PF745) from a cell lysate.

Cation exchange chromatography was tested as a third chromatography step for the purification of PF745, following both HIC and AEX chromatography steps.

### Resin Screening

The CEX resins used in a third chromatography step are shown in Table 8.

TABLE 8
Cation exchange chromatography
resins that may be used for the third
chromatography step

| Resin | Manufacturer | Catalog # |
|---|---|---|
| POROS XS | Thermo Fisher | 4404336 |
| Capto MMC | GE Healthcare | 17-5317-99 |
| MX-TRP-650M | Tosoh | 0022817 |
| CMM HyperCel | Pall | 20270-025 |
| CMM HyperCel | Pall | PRCCMMHCEL1ML |
| (pre-packed | | |
| column) | | |
| Sulfate-650F | Tosoh | 0023467 |
| NH2-750F | Tosoh | 0023438 |
| CaPure-HA | Tosoh | 45039 |
| PPG-600M | Tosoh Bioscience | 0021301 |

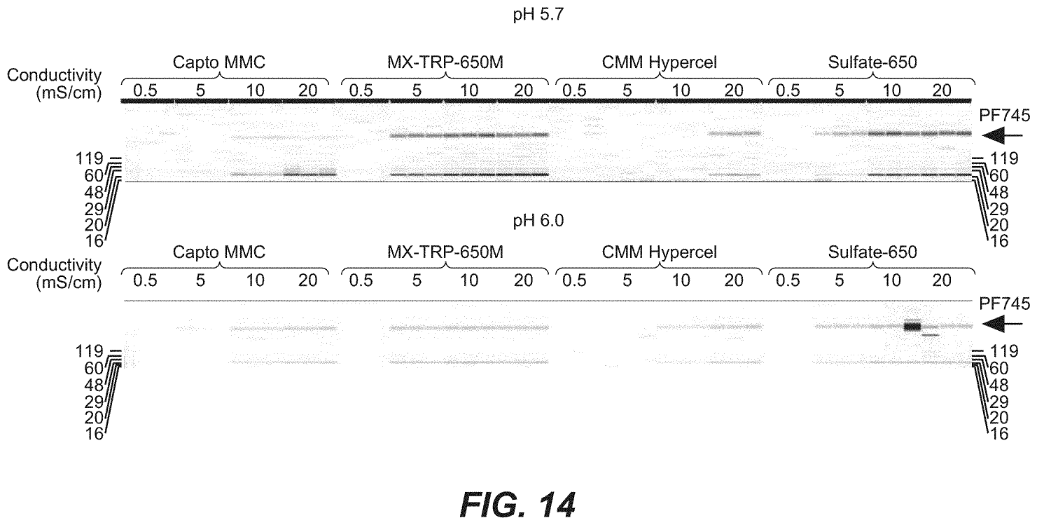

Four mixed-mode resins (Capto MMC, MX-Trp-650M, CMM HyperCel, Sulfate-650F) were screened to see if they would bind a target (e.g., PF745) at higher conductivity (5-10 mS/cm) for higher resolution and purity. Flowthrough purification was performed using 96-well filter plates (Agilent, Cat# 200957-100) and the Biosero Automation System, which includes a Tecan Freedom Evo 200 liquid handling system and a Bionex HiG4 automated centrifuge. FIG. 14 shows the SDS-CGE image of flow-through fractions from the eight combinations of pH and conductivity load, subjected to four different mixed-mode resins. The absence of a significant PF745 band in all eight load conditions for Capto MMC is evidence of good binding. CMM HyperCel demonstrated similar performance, except for breakthrough at pH 5.7 and 20 mS/cm. MX-TRP-650M and Sulfate-650F demonstrated significantly lower binding capacity, as evidenced by significant PF745 bands at ≥5 mS/cm.

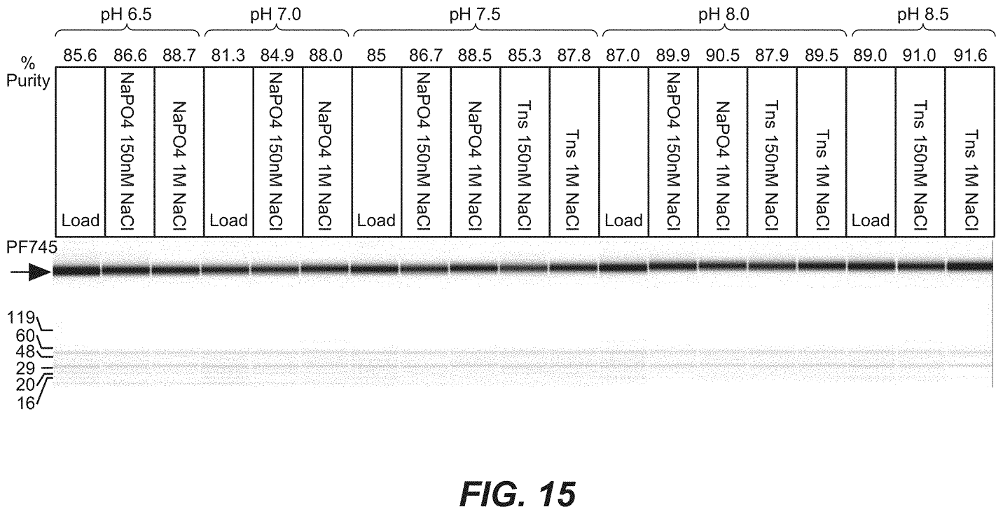

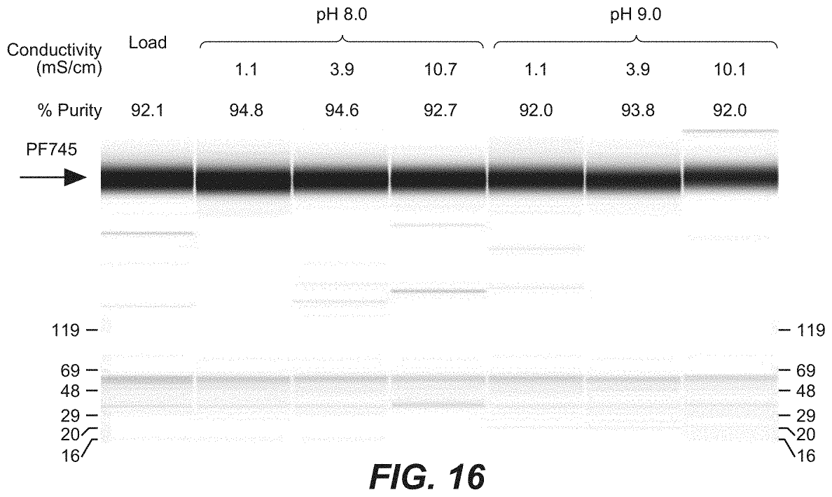

Four additional resins were evaluated in batch mixing studies as third column candidates (Capto Core 400, TOYOPEARL NH2-750F, CaPure-HA, and TOYOPEARL PPG-600M). An SDS-CGE image of Capto Core 400 load and flow-through fractions demonstrates no significant increase in purity in any condition (FIG. 15). Insufficient binding of LMW impurities (<69 kDa) was observed. An SDS-CGE image of NH2-750F load and flow-through fractions demonstrates no significant increase in purity in any condition tested (FIG. 16). No binding of LMW impurities (<69 kDa) was observed.

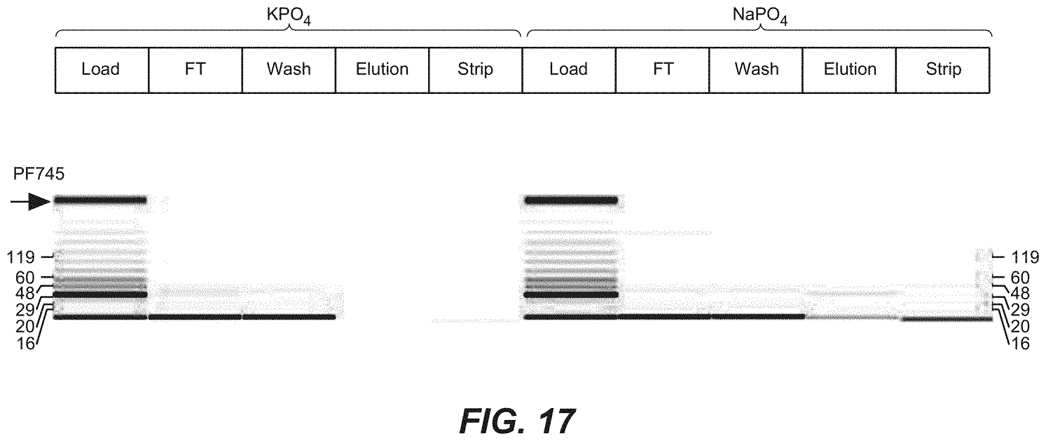

FIG. 17 shows the SDS-CGE image of CaPure-HA fractions from batch-mixing. The flow-through does not display a significant PF745 band and measured at near zero concentration, indicating good binding. The absence of PF745 bands in the wash, elution, and strip fractions indicated that the bound PF745 was not recovered. The concentration by UV shows <10% recovery in the elution fractions and negligible recovery in the strip.

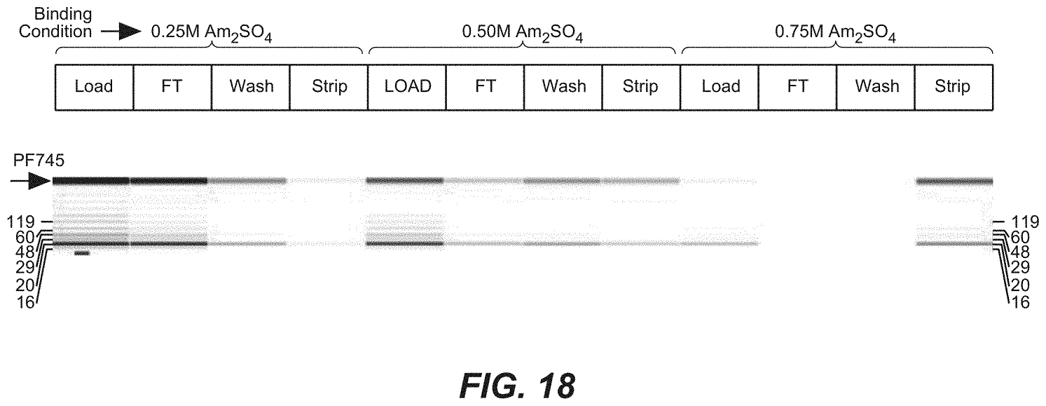

Finally, FIG. 18 shows an SDS-CGE image of PPG-600M fractions. The 0.75 M ammonium sulfate load adjustment precipitated PF745 as evidenced by an absence of bands by SDS-CGE. SDS-CGE integration of the 0.25 M ammonium sulfate load condition results in load purity of 49.6% compared to 55.1% in the flow-through; the small increase in purity is likely an artifact of low concentration in the flow-through fraction, causing LMW bands to fall below the limit of quantitation. No significant purity improvement was observed in the flow-through fraction of the 0.25 M ammonium sulfate load condition. At 0.5 M ammonium sulfate loading, the lower PF745 band intensity in flow-through compared to load indicated significant binding. The stronger intensity in the wash fraction indicated that 0.5 M ammonium sulfate may not strongly promote binding, and the transition to wash buffer elutes the protein. Additionally, a PF745 band was present in the strip, indicating that it would be difficult to achieve good recovery from the 0.5 M ammonium sulfate load condition.

Three resins, POROS XS, CMM HyperCel, and Capto MMC, were scaled to 0.66 cm diameter columns. A dynamic binding capacity (DBC) test at 15 mg/mL resin challenge showed no significant breakthrough in the flow-through in the chromatogram or SDS-CGE results. Recovery was high (86%) and RP-HPLC purity was in line with previous results (98.9% purity). These results support loading up to 15 mg/mL of protein for CEX chromatography. A safety factor of 20% was applied to set the load challenge limit at 12 mg/mL. Mixed-mode and gel filtration resin screening experiments demonstrated efficient purification using the POROS XS resin.

When the pre-elution wash step was removed, recovery was improved by 20-30%, while maintaining product quality comparable to runs with the pre-elution wash. The CEX step, e.g., using a POROS XS resin, step significantly improved RP-HPLC purity and SDS-CGE purity and reduced HCP and HCDNA levels.

A DBC run identified that load challenge could be increased from 5 g/L to 12 g/L with similar purification results. Effective CEX chromatography conditions, using POROS XS as an exemplary column, included, 1) load conductivity: 1.0±0.1 mS/cm (achieved by UF/DF 3); 2) elution NaCl concentration: 8.35±0.08 mM; 3) load challenge: ≤12 g/L; and, 4) load concentration: ≤6 mg/mL. These conditions are expected to generate a recovery of ≥70%, ≥97% RP-HPLC purity, and ≥99% SE-HPLC purity. Table 9 shows an exemplary CEX chromatography method that follows UF/DF 3.

TABLE 9
Exemplary CEX chromatography method using POROS XS

| | | | | LFR |
|---|---|---|---|---|
| Phase | Buffer/Solution | CV | | (cm/h) |
| Pre-EQ | 50 mM Tris, 3M | 3 | | 120 |
| | NaCl, pH 8.0 | | | |
| Equilibration | 20 mM MES, | 4 | | 120 |
| | 1 mM EDTA, | | | |
| | 2.7 mM NaCl, | | | |
| | pH 6.0, 1.0 mS/cm | | | |
| Load | UF/DF 3 intermediate | Challenge: | | 55 |
| | adjusted to | 5-12 | mg/mL | |
| | pH 6.0 ± 0.1, | | resin | |
| | 1.0 ± 0.1 mS/cm, | | | |
| | 4-6 mg/mL | | | |
| Post-Load | Same as EQ | 1 | | 55 |
| Wash | | 3 | | 120 |
| Isocratic | 20 mM MES, | 5 | | 120 |
| Elution | 1 mM EDTA, | | | |
| | 8.35 mM NaCl, | | | |
| | pH 6.2, 1.9 mS/cm | | | |
| Strip | 50 mM Tris, 3M | 3 | | 120 |
| | NaCl, pH 8.0 | | | |
| Sanitization | 1N NaOH | 3 | (upflow) | 120 |
| | | | | (+60-minute |
| | | | | hold) |
| Storage | 0.1N NaOH | 3 | (upflow) | 120 |

### Conclusion

A CEX chromatography step, following a HIC step and AEX step, further improved the purity of the charge-shield protein PF745.

1. A method of purifying a charge-shielded fusion protein from a cell lysate or periplasmic releasate, wherein the charge-shielded fusion protein comprises a biologically active domain and a charge-shielding domain, and wherein the method comprises hydrophobic interaction chromatography as a first chromatography step.

2. A method for producing a charge-shielded fusion protein from a cell lysate or periplasmic releasate wherein the charge-shielded fusion protein comprises a biologically active domain and a charge-shielding domain, wherein the method comprises
i) culturing cells comprising a nucleic acid encoding the charge-shielded fusion protein; and
ii) purifying the charge-shielded fusion protein, wherein the charge-shielded protein is purified from the cell lysate or periplasmic releasate using hydrophobic interaction chromatography as a first chromatography step.

3. The method of claim 1, wherein the charge-shielded fusion protein is at least 45% pure after the first chromatography step.

4. The method of claim 1, wherein the method further comprises an anion exchange chromatography.

5. The method of claim 1, wherein the method further comprises a cation exchange chromatography.

6. The method of claim 1, wherein the method comprises a sequence of chromatography steps comprising in order:
i) hydrophobic interaction chromatography;
ii) anion exchange chromatography; and
iii) cation exchange chromatography.

7. The method of claim 1, wherein the biologically active domain is charged at pH of about 7.0, wherein the charge-shielding domain increases the hydrodynamic radius of the protein, and/or wherein the charge-shielding domain does not have a charge at pH of about 7.0.

8. The method of claim 1, wherein the molecular weight of the biologically active domain is less than the molecular weight of the charge-shielding domain.

9. The method of claim 1, wherein the molecular weight of the charge-shielding domain is between 10 kDa and 60 kDa.

10-13. (canceled)

14. The method of claim 1, wherein the charge-shielding domain has a random coil or disordered structure.

15. The method of claim 1, wherein the charge-shielding domain is a polypeptide consisting of one or more of alanine, serine and proline residues.

16. The method of claim 15, wherein the charge-shielding domain is a polypeptide consisting of proline and alanine residues.

17. A method for producing a PASylated biologically active fusion protein from a cell lysate or periplasmic releasate comprising
i) culturing cells comprising a nucleic acid encoding the PASylated biologically active protein; and
ii) purifying the PASylated biologically active protein,
wherein the PASylated biologically active protein is purified from the cell lysate or periplasmic releasate using hydrophobic interaction chromatography as a first chromatography step.

18. A method for purifying a charge-shielded fusion protein comprising a biologically active domain and a charge-shielding domain from a cell lysate or periplasmic releasate, the method comprising the following steps in order
i) applying a load solution comprising the charge-shielded fusion protein to a hydrophobic interaction chromatography column;
ii) applying a wash solution to the hydrophobic interaction chromatography column;
iii) applying an elution solution to the hydrophobic interaction column to elute the charge-shielded protein;
iv) applying the eluted charge-shielded fusion protein in iii) as a load solution to an anion exchange chromatography column;
v) eluting the charge-shielded fusion protein from the anion exchange chromatography column;
vi) applying the eluted charge-shielded fusion protein in v) as a load solution to a cation exchange chromatography column;
vii) applying a wash solution to the cation exchange chromatography column;
viii) applying an elution solution to the cation exchange chromatography column to elute the charge-shielded fusion protein.

19-31. (canceled)

32. The method of claim 1, wherein the biologically active domain is an asparaginase subunit.

33. The method of claim 32, wherein the asparaginase is selected from the group consisting of an E. coli asparaginase and an Erwinia asparaginase.

34-35. (canceled)

36. The method of claim 2, wherein the cell is a bacterial cell.

37. The method of claim 36, wherein the cell is an E. coli cell or a Pseudomonas cell.

38. A charge-shielded protein produced by the method of claim 1.

39. A pharmaceutical composition comprising the charge-shielded protein of claim 38 and a pharmaceutically acceptable carrier.

40. A method of treatment comprising administering a composition comprising the charge-shielded protein of claim 38 to an individual in need thereof.

41. A composition comprising a PASylated asparaginase, wherein the PASylated asparaginase is at least 45% pure.
